SEQUENCE LISTING (91 sequences)

SEQ ID NO: 1 - DNA, 40 bp, Artificial Sequence
  source 1..40: /mol_type=“unassigned DNA” /note=“Left HsInv030_MLPA_INV probe ” /organism=“Artificial Sequence”
gggtagggaatcccttggaccttccccttccctccatgaa

SEQ ID NO: 2 - DNA, 46 bp, Artificial Sequence
  source 1..46: /mol_type=“unassigned DNA” /note=“Left HsInv030_MLPA_REF probe” /organism=“Artificial Sequence”
gggtagggaatcccttggacattcaggggttccaagcaccctgaag

SEQ ID NO: 3 - DNA, 59 bp, Artificial Sequence
  source 1..59: /mol_type=“unassigned DNA” /note=“Left HsInv031_MLPA_INV probe” /organism=“Artificial Sequence”
gggaaccgtagcacatggaccttgcgctggatctttgctggtgttttgctcatgtattg

SEQ ID NO: 4 - DNA, 62 bp, Artificial Sequence
  source 1..62: /mol_type=“unassigned DNA” /note=“Left HsInv031_MLPA_REF_2 probe” /organism=“Artificial Sequence”
gggaaccgtagcacatggacctggagcgacctgtgagatagaacaaattctctccatgtttg

SEQ ID NO: 5 - DNA, 83 bp, Artificial Sequence
  source 1..83: /mol_type=“unassigned DNA” /note=“Left HsInv040_MLPA_INV probe” /organism=“Artificial Sequence”
gggtagggaatcccttggacctatagcgacttacggacggcgtattcgtactgactgcccggtcttgaaaatgttgcttaagc

SEQ ID NO: 6 - DNA, 55 bp, Artificial Sequence
  source 1..55: /mol_type=“unassigned DNA” /note=“Left HsInv040_MLPA_REF probe” /organism=“Artificial Sequence”
gggtagggaatcccttggacctccccattgacaagagagtcaatttgtcctctga

SEQ ID NO: 7 - DNA, 54 bp, Artificial Sequence
  source 1..54: /mol_type=“unassigned DNA” /note=“Left HsInv045_MLPA_INV_2 probe ” /organism=“Artificial Sequence”
gggaaccgtagcacatggacctatagcgactccagccccctatgtgggtttcta

SEQ ID NO: 8 - DNA, 66 bp, Artificial Sequence
  source 1..66: /mol_type=“unassigned DNA” /note=“Left HsInv045_MLPA_REF_2 probe” /organism=“Artificial Sequence”
gggaaccgtagcacatggacctatagcgactgcatcccacttttggaatgccatattctagagctc

SEQ ID NO: 9 - DNA, 55 bp, Artificial Sequence
  source 1..55: /mol_type=“unassigned DNA” /note=“Left HsInv055_MLPA_INV probe” /organism=“Artificial Sequence”
gggaaccgtagcacatggacttcttagcagagctcgagcactgtgctgggggatc

SEQ ID NO: 10 - DNA, 56 bp, Artificial Sequence
  source 1..56: /mol_type=“unassigned DNA” /note=“Left HsInv055_MLPA_INV_bis probe” /organism=“Artificial Sequence”
gggaaccgtagcacatggacctatagtcagtcaggaggcatgagggtcagggatcc

SEQ ID NO: 11 - DNA, 58 bp, Artificial Sequence
  source 1..58: /mol_type=“unassigned DNA” /note=“Left HsInv055_MLPA_REF probe” /organism=“Artificial Sequence”
gggaaccgtagcacatggacctaaagccagggagccaagtggtcttgctcagtggatc

SEQ ID NO: 12 - DNA, 44 bp, Artificial Sequence
  source 1..44: /mol_type=“unassigned DNA” /note=“Left HsInv061_MLPA_INV probe ” /organism=“Artificial Sequence”
gggtagggaatcccttggagacgtgtagggcttgcaggcatgga

SEQ ID NO: 13 - DNA, 50 bp, Artificial Sequence
  source 1..50: /mol_type=“unassigned DNA” /note=“Left HsInv061_MLPA_REF probe” /organism=“Artificial Sequence”
gggtagggaatcccttggaccatgaggtggtggttgcagtgagccgagat

SEQ ID NO: 14 - DNA, 70 bp, Artificial Sequence
  source 1..70: /mol_type=“unassigned DNA” /note=“Left HsInv072_MLPA_INV probe” /organism=“Artificial Sequence”
gggtagggaatcccttggacctatagcgacttacggacggcgtaccccttatgtgggcttaccgaagctt

SEQ ID NO: 15 - DNA, 95 bp, Artificial Sequence
  source 1..95: /mol_type=“unassigned DNA” /note=“Left HsInv072_MLPA_REF probe” /organism=“Artificial Sequence”
gggtagggaatcccttggacctatagcgacttacggacggcgtatccgacctgtatcctgagactttgctgaagttgcttatcagcttaagaagc

SEQ ID NO: 16 - DNA, 77 bp, Artificial Sequence
  source 1..77: /mol_type=“unassigned DNA” /note=“Left HsInv114_MLPA_INV_2 probe ” /organism=“Artificial Sequence”
gggaaccgtagcacatggacctatagcgacttacggacggcgtatccgacctgacttatggaacgaatgagtcagtg

SEQ ID NO: 17 - DNA, 85 bp, Artificial Sequence
  source 1..85: /mol_type=“unassigned DNA” /note=“Left HsInv114_MLPA_REF_2 probe” /organism=“Artificial Sequence”
gggaaccgtagcacatggacctatagcgacttacggacggcgtatccgactccttgcctcacatgctcaagacaacaacccttgg

SEQ ID NO: 18 - DNA, 52 bp, Artificial Sequence
  source 1..52: /mol_type=“unassigned DNA” /note=“Left HsInv124_MLPA_COM_2 probe” /organism=“Artificial Sequence”
gggttccctaagggttggacctatactctagggccccactggccaaaagctt

SEQ ID NO: 19 - DNA, 65 bp, Artificial Sequence
  source 1..65: /mol_type=“unassigned DNA” /note=“Left HsInv209_MLPA_INV probe” /organism=“Artificial Sequence”
gggttccctaagggttggacctatagcgactatacatcattcccacaggaatgtgccaagagaag

SEQ ID NO: 20 - DNA, 68 bp, Artificial Sequence
  source 1..68: /mol_type=“unassigned DNA” /note=“Left HsInv209_MLPA_REF probe” /organism=“Artificial Sequence”
gggttccctaagggttggacctatagcgactatacacaaggttgcatcgtgaccacgggcctggaaag

SEQ ID NO: 21 - DNA, 68 bp, Artificial Sequence
  source 1..68: /mol_type=“unassigned DNA” /note=“Left HsInv278_MLPA_INV probe” /organism=“Artificial Sequence”
gggttccctaagggttggacctatagcgacttacggacgacgtatacgctgcctttgctcgcagatct

SEQ ID NO: 22 - DNA, 71 bp, Artificial Sequence
  source 1..71: /mol_type=“unassigned DNA” /note=“Left HsInv278_MLPA_REF probe” /organism=“Artificial Sequence”
gggttccctaagggttggacctatagcgacttacggacggcgtacatggatgcagctcttgtcctaagaga

SEQ ID NO: 23 - DNA, 49 bp, Artificial Sequence
  source 1..49: /mol_type=“unassigned DNA” /note=“Left HsInv340_MLPA_INV_2 probe” /organism=“Artificial Sequence”
gggttccctaagggttggacatccatatcagttttgggttggagggatg

SEQ ID NO: 24 - DNA, 59 bp, Artificial Sequence
  source 1..59: /mol_type=“unassigned DNA” /note=“Left HsInv340_MLPA_REF probe” /organism=“Artificial Sequence”
gggttccctaagggttggacctatagcggtaagtatgacattacatgtttcttggatcc

SEQ ID NO: 25 - DNA, 71 bp, Artificial Sequence
  source 1..71: /mol_type=“unassigned DNA” /note=“Left HsInv341_MLPA_INV probe” /organism=“Artificial Sequence”
gggtagggaatcccttggacctatagcgacttacggaccggttccatggtcaagaatttgaaaagagatgc

SEQ ID NO: 26 - DNA, 74 bp, Artificial Sequence
  source 1..74: /mol_type=“unassigned DNA” /note=“Left HsInv341_MLPA_REF probe” /organism=“Artificial Sequence”
gggtagggaatcccttggacctatagcgacttacggacggcgtattatcatagtggcagggcaggatgctatgc

SEQ ID NO: 27 - DNA, 75 bp, Artificial Sequence
  source 1..75: /mol_type=“unassigned DNA” /note=“Left HsInv344_MLPA_INV probe” /organism=“Artificial Sequence”
gggttccctaagggttggacctatagcgacttacggacggactagtagctgggattacaggtgcacgtcaccaag

SEQ ID NO: 28 - DNA, 57 bp, Artificial Sequence
  source 1..57: /mol_type=“unassigned DNA” /note=“Left HsInv344_MLPA_REF_2 probe” /organism=“Artificial Sequence”
gggttccctaagggttggacctaagcacatgagggtcttgtagacaccacagtaaag

SEQ ID NO: 29 - DNA, 73 bp, Artificial Sequence
  source 1..73: /mol_type=“unassigned DNA” /note=“Left HsInv347_MLPA_INV probe” /organism=“Artificial Sequence”
gggtagggaatcccttggacctatagcgacttacggacggcgccccatcaaaagaataactgcagggatggga

SEQ ID NO: 30 - DNA, 76 bp, Artificial Sequence
  source 1..76: /mol_type=“unassigned DNA” /note=“Left HsInv347_MLPA_REF probe ” /organism=“Artificial Sequence”
gggtagggaatcccttggacctatagcgacttacggacggcgtattgcgaggtgtttccctcttccctgattatga

SEQ ID NO: 31 - DNA, 63 bp, Artificial Sequence
  source 1..63: /mol_type=“unassigned DNA” /note=“Left HsInv374_MLPA_INV probe ” /organism=“Artificial Sequence”
gggaaccgtagcacatggaccgccggcctacttactttgtatataaatgtgtaaactcctcaa

SEQ ID NO: 32 - DNA, 66 bp, Artificial Sequence
  source 1..66: /mol_type=“unassigned DNA” /note=“Left HsInv374_MLPA_REF probe” /organism=“Artificial Sequence”
gggaaccgtagcacatggaccgccgtcgggacgttgaactaatttccttattggagttcattattg

SEQ ID NO: 33 - DNA, 45 bp, Artificial Sequence
  source 1..45: /mol_type=“unassigned DNA” /note=“Left HsInv379_MLPA_INV probe” /organism=“Artificial Sequence”
gggaaccgtagcacatggaccctgctgcagttacatgagaggatc

SEQ ID NO: 34 - DNA, 47 bp, Artificial Sequence
  source 1..47: /mol_type=“unassigned DNA” /note=“Left HsInv379_MLPA_REF probe” /organism=“Artificial Sequence”
gggaaccgtagcacatggacctgtgacctgctgcagttacatgagag

SEQ ID NO: 35 - DNA, 40 bp, Artificial Sequence
  source 1..40: /mol_type=“unassigned DNA” /note=“Left HsInv389_MLPA_INV probe” /organism=“Artificial Sequence”
gggttccctaagggttggaccagccctgcctccacaaatg

SEQ ID NO: 36 - DNA, 41 bp, Artificial Sequence
  source 1..41: /mol_type=“unassigned DNA” /note=“Left HsInv389_MLPA_REF probe” /organism=“Artificial Sequence”
gggttccctaagggttggacctgggattggcaccttgaatg

SEQ ID NO: 37 - DNA, 79 bp, Artificial Sequence
  source 1..79: /mol_type=“unassigned DNA” /note=“Left HsInv393_MLPA_INV probe” /organism=“Artificial Sequence”
gggttccctaagggttggacctatagcgacttacggacggcctggctgaactcatagtgttaggtgtcagatgactgag

SEQ ID NO: 38 - DNA, 83 bp, Artificial Sequence
  source 1..83: /mol_type=“unassigned DNA” /note=“Left HsInv393_MLPA_REF probe” /organism=“Artificial Sequence”
gggttccctaagggttggacctatagcgacttacggacggcgtattcgtcagcatctcacaaagaccaattgtcaatacgtag

SEQ ID NO: 39 - DNA, 59 bp, Artificial Sequence
  source 1..59: /mol_type=“unassigned DNA” /note=“Left HsInv396_MLPA_INV probe” /organism=“Artificial Sequence”
gggtagggaatcccttggacctatagcgaccgttgaatttgattttgggtctcagccac

SEQ ID NO: 40 - DNA, 65 bp, Artificial Sequence
  source 1..65: /mol_type=“unassigned DNA” /note=“Left HsInv396_MLPA_REF probe” /organism=“Artificial Sequence”
gggtagggaatcccttggacctatagcgactatacacgttgaatttgattttgggtctcagccac

SEQ ID NO: 41 - DNA, 62 bp, Artificial Sequence
  source 1..62: /mol_type=“unassigned DNA” /note=“Left HsInv397_MLPA_INV_2 probe” /organism=“Artificial Sequence”
gggaaccgtagcacatggacctgtagcgacttagaattggctatggggaaataactgagctc

SEQ ID NO: 42 - DNA, 65 bp, Artificial Sequence
  source 1..65: /mol_type=“unassigned DNA” /note=“Left HsInv397_MLPA_REF_2 probe ” /organism=“Artificial Sequence”
gggaaccgtagcacatggaccttgatcttggatgaggccaccctcaaggctgagacccagagctc

SEQ ID NO: 43 - DNA, 78 bp, Artificial Sequence
  source 1..78: /mol_type=“unassigned DNA” /note=“Left HsInv403_MLPA_INV probe” /organism=“Artificial Sequence”
gggtagggaatcccttggacaccctccctgtggagagactgtcgtcagaccaactcaaaattacaaagttttccaaag

SEQ ID NO: 44 - DNA, 73 bp, Artificial Sequence
  source 1..73: /mol_type=“unassigned DNA” /note=“Left HsInv403_MLPA_REF probe” /organism=“Artificial Sequence”
gggtagggaatcccttggacctatagcgacttacggacggcgtattcctgcatttcagtgttaaggcccagaa

SEQ ID NO: 45 - DNA, 48 bp, Artificial Sequence
  source 1..48: /mol_type=“unassigned DNA” /note=“Left HsInv790_MLPA_INV probe” /organism=“Artificial Sequence”
gggaaccgtagcacatggacctggcagactgtccagataggaaccttg

SEQ ID NO: 46 - DNA, 52 bp, Artificial Sequence
  source 1..52: /mol_type=“unassigned DNA” /note=“Left HsInv790_MLPA_REF probe” /organism=“Artificial Sequence”
gggaaccgtagcacatggacctatgaggatcaggcaaaggggaaattggatc

SEQ ID NO: 47 - DNA, 50 bp, Artificial Sequence
  source 1..50: /mol_type=“unassigned DNA” /note=“Left HsInv832_MLPA_INV probe” /organism=“Artificial Sequence”
gggtagggaatcccttggacgacttttgtatcaggtgtaaggatgggatc

SEQ ID NO: 48 - DNA, 53 bp, Artificial Sequence
  source 1..53: /mol_type=“unassigned DNA” /note=“Left HsInv832_MLPA_REF probe” /organism=“Artificial Sequence”
gggtagggaatcccttggacggctagccatatgtagaaagctgaaactggatc

SEQ ID NO: 49 - DNA, 51 bp, Artificial Sequence
  source 1..51: /mol_type=“unassigned DNA” /note=“Right HsInv030_MLPA_INV probe” /organism=“Artificial Sequence”
gcttgcctcctgaaatacttttatgagctctagattggatcttgctggcac

SEQ ID NO: 50 - DNA, 48 bp, Artificial Sequence
  source 1..48: /mol_type=“unassigned DNA” /note=“Right HsInv030_MLPA_REF probe” /organism=“Artificial Sequence”
cttcatggagggaaggggaaggctctctagattggatcttgctggcac

SEQ ID NO: 51 - DNA, 45 bp, Artificial Sequence
  source 1..45: /mol_type=“unassigned DNA” /note=“Right HsInv031_MLPA_COM_2 probe” /organism=“Artificial Sequence”
aattccctcctcctgggagaggtctagattggatcttgctggcac

SEQ ID NO: 52 - DNA, 56 bp, Artificial Sequence
  source 1..56: /mol_type=“unassigned DNA” /note=“Right HsInv040_MLPA_INV probe” /organism=“Artificial Sequence”
ttcagaggacaaattgactctcttgtcaatggctctagattggatcttgctggcac

SEQ ID NO: 53 - DNA, 87 bp, Artificial Sequence
  source 1..87: /mol_type=“unassigned DNA” /note=“Right HsInv040_MLPA_REF probe” /organism=“Artificial Sequence”
agcttaatttaatacttacttttactagcttattataaaggatacatctcaggaacagcgcccctctagattggatcttgctggcac

SEQ ID NO: 54 - DNA, 56 bp, Artificial Sequence
  source 1..56: /mol_type=“unassigned DNA” /note=“Right HsInv045_MLPA_INV_2 probe” /organism=“Artificial Sequence”
gagctcttcgtaaattagcctgtctagaaattctctagattggatcttgctggcac

SEQ ID NO: 55 - DNA, 47 bp, Artificial Sequence
  source 1..47: /mol_type=“unassigned DNA” /note=“Right HsInv045_MLPA_REF_2 probe” /organism=“Artificial Sequence”
tagaaacccacatagggggctgggtctagattggatcttgctggcac

SEQ ID NO: 56 - DNA, 61 bp, Artificial Sequence
  source 1..61: /mol_type=“unassigned DNA” /note=“Right HsInv055_MLPA_COM probe” /organism=“Artificial Sequence”
cagaggccagcccaagtggctgcctagttctcttagactctagattggatcttgctggcac

SEQ ID NO: 57 - DNA, 53 bp, Artificial Sequence
  source 1..53: /mol_type=“unassigned DNA” /note=“Right HsInv061_MLPA_INV probe” /organism=“Artificial Sequence”
agatctcggctcactgcaaccaccacctcctctagattggatcttgctggcac

SEQ ID NO: 58 - DNA, 50 bp, Artificial Sequence
  source 1..50: /mol_type=“unassigned DNA” /note=“Right HsInv061_MLPA_REF probe ” /organism=“Artificial Sequence”
ctgtctgaggccaaagtctacaacttctctagattggatcttgctggcac

SEQ ID NO: 59 - DNA, 75 bp, Artificial Sequence
  source 1..75: /mol_type=“unassigned DNA” /note=“Right HsInv072_MLPA_INV probe” /organism=“Artificial Sequence”
cttaagctgataagcaacttcagcaaagtctcaggatacagaatcaatgtgctctagattggatcttgctggcac

SEQ ID NO: 60 - DNA, 53 bp, Artificial Sequence
  source 1..53: /mol_type=“unassigned DNA” /note=“Right HsInv072_MLPA_REF probe” /organism=“Artificial Sequence”
ttctatgccacagaggcaaatcagcattcctctagattggatcttgctggcac

SEQ ID NO: 61 - DNA, 57 bp, Artificial Sequence
  source 1..57: /mol_type=“unassigned DNA” /note=“Right HsInv114_MLPA_INV_2 probe” /organism=“Artificial Sequence”
gatcctctcaagggagagcccaaggctggtgttctctagattggatcttgctggcac

SEQ ID NO: 62 - DNA, 52 bp, Artificial Sequence
  source 1..52: /mol_type=“unassigned DNA” /note=“Right HsInv114_MLPA_REF_2 probe” /organism=“Artificial Sequence”
gatccactgactcattcgttccataagtctctagattggatcttgctggcac

SEQ ID NO: 63 - DNA, 50 bp, Artificial Sequence
  source 1..50: /mol_type=“unassigned DNA” /note=“Right HsInv124_MLPA_INV_2 probe” /organism=“Artificial Sequence”
ctttaaatcacgggcagtttaggaaggtctagattggatcttgctggcac

SEQ ID NO: 64 - DNA, 53 bp, Artificial Sequence
  source 1..53: /mol_type=“unassigned DNA” /note=“Right HsInv124_MLPA_REF probe” /organism=“Artificial Sequence”
ccaaaataccttccacgggaaattcaagcctctagattggatcttgctggcac

SEQ ID NO: 65 - DNA, 49 bp, Artificial Sequence
  source 1..49: /mol_type=“unassigned DNA” /note=“Right HsInv209_MLPA_COM probe” /organism=“Artificial Sequence”
cttcccaggtgagctgagtcttatcctctagattggatcttgctggcac

SEQ ID NO: 66 - DNA, 52 bp, Artificial Sequence
  source 1..52: /mol_type=“unassigned DNA” /note=“Right HsInv278_MLPA_INV probe” /organism=“Artificial Sequence”
cttaggacaagagctgcatccatggacagtctagattggatcttgctggcac

SEQ ID NO: 67 - DNA, 52 bp, Artificial Sequence
  source 1..52: /mol_type=“unassigned DNA” /note=“Right HsInv278_MLPA_REF probe” /organism=“Artificial Sequence”
tcttgtcataaacacagatcccaggctgctctagattggatcttgctggcac

SEQ ID NO: 68 - DNA, 59 bp, Artificial Sequence
  source 1..59: /mol_type=“unassigned DNA” /note=“Right HsInv340_MLPA_INV_2 probe” /organism=“Artificial Sequence”
gatccaagaaacatgtaatgtcatacttacctaatctctagattggatcttgctggcac

SEQ ID NO: 69 - DNA, 52 bp, Artificial Sequence
  source 1..52: /mol_type=“unassigned DNA” /note=“Right HsInv340_MLPA_REF probe” /organism=“Artificial Sequence”
tcatgccttctagtttgtagggtttctgctctagattggatcttgctggcac

SEQ ID NO: 70 - DNA, 50 bp, Artificial Sequence
  source 1..50: /mol_type=“unassigned DNA” /note=“Right HsInv341_MLPA_COM probe” /organism=“Artificial Sequence”
attcagccagtcattcatgatgttccctctagattggatcttgctggcac

SEQ ID NO: 71 - DNA, 56 bp, Artificial Sequence
  source 1..56: /mol_type=“unassigned DNA” /note=“Right HsInv344_MLPA_INV probe” /organism=“Artificial Sequence”
ctttactgtggtgtctacaagaccctcatgatctctagattggatcttgctggcac

SEQ ID NO: 72 - DNA, 77 bp, Artificial Sequence
  source 1..77: /mol_type=“unassigned DNA” /note=“Right HsInv344_MLPA_REF_2 probe” /organism=“Artificial Sequence”
cttctttaggcagaatgaatgttttaaagtttaagaataggatctgctgacagctctagattggatcttgctggcac

SEQ ID NO: 73 - DNA, 54 bp, Artificial Sequence
  source 1..54: /mol_type=“unassigned DNA” /note=“Right HsInv347_MLPA_COM probe” /organism=“Artificial Sequence”
attctctttcaggcatgtgatttcataggactctagattggatcttgctggcac

SEQ ID NO: 74 - DNA, 65 bp, Artificial Sequence
  source 1..65: /mol_type=“unassigned DNA” /note=“Right HsInv374_MLPA_COM probe” /organism=“Artificial Sequence”
gaattctaatattactcctaaagggaaaaatctatgggcgcctctagattggatcttgctggcac

SEQ ID NO: 75 - DNA, 47 bp, Artificial Sequence
  source 1..47: /mol_type=“unassigned DNA” /note=“Right HsInv379_MLPA_INV probe ” /organism=“Artificial Sequence”
ccaagcaaatcacagcggccctactctagattggatcttgctggcac

SEQ ID NO: 76 - DNA, 48 bp, Artificial Sequence
  source 1..48: /mol_type=“unassigned DNA” /note=“Right HsInv379_MLPA_REF probe” /organism=“Artificial Sequence”
gatccacaggcagatgcagttaaggtctagattggatcttgctggcac

SEQ ID NO: 77 - DNA, 50 bp, Artificial Sequence
  source 1..50: /mol_type=“unassigned DNA” /note=“Right HsInv389_MLPA_COM probe” /organism=“Artificial Sequence”
catggaggacaggcgatggggtctaactctagattggatcttgctggcac

SEQ ID NO: 78 - DNA, 57 bp, Artificial Sequence
  source 1..57: /mol_type=“unassigned DNA” /note=“Right HsInv393_MLPA_INV probe” /organism=“Artificial Sequence”
atctacgtattgacaattggtctttgtgagatgctctagattggatcttgctggcac

SEQ ID NO: 79 - DNA, 57 bp, Artificial Sequence
  source 1..57: /mol_type=“unassigned DNA” /note=“Right HsInv393_MLPA_REF probe” /organism=“Artificial Sequence”
atctgtgggaaagtcaaatctttttgatccagcctctagattggatcttgctggcac

SEQ ID NO: 80 - DNA, 57 bp, Artificial Sequence
  source 1..57: /mol_type=“unassigned DNA” /note=“Right HsInv396_MLPA_INV probe” /organism=“Artificial Sequence”
gaattcatattcacaataaatattccaagacccctctagattggatcttgctggcac

SEQ ID NO: 81 - DNA, 53 bp, Artificial Sequence
  source 1..53: /mol_type=“unassigned DNA” /note=“Right HsInv396_MLPA_REF probe” /organism=“Artificial Sequence”
gaattcaatagaatattaagagccagagcctctagattggatcttgctggcac

SEQ ID NO: 82 - DNA, 60 bp, Artificial Sequence
  source 1..60: /mol_type=“unassigned DNA” /note=“Right HsInv397_MLPA_COM_2 probe” /organism=“Artificial Sequence”
aaaacacaaatccgttgaggttcagaatcccagagactctagattggatcttgctggcac

SEQ ID NO: 83 - DNA, 57 bp, Artificial Sequence
  source 1..57: /mol_type=“unassigned DNA” /note=“Right HsInv403_MLPA_INV probe” /organism=“Artificial Sequence”
cttgaataagtgaaattacttgctgggatgtttgtctagattggatcttgctggcac

SEQ ID NO: 84 - DNA, 63 bp, Artificial Sequence
  source 1..63: /mol_type=“unassigned DNA” /note=“Right HsInv403_MLPA_REF probe” /organism=“Artificial Sequence”
agctttggaaaactttgtaattttgagttggtctgacgactctagattggatcttgctggcac

SEQ ID NO: 85 - DNA, 50 bp, Artificial Sequence
  source 1..50: /mol_type=“unassigned DNA” /note=“Right HsInv790_MLPA_INV probe ” /organism=“Artificial Sequence”
gatccaatccgtagtcttttgtccctctctagattggatcttgctggcac

SEQ ID NO: 86 - DNA, 49 bp, Artificial Sequence
  source 1..49: /mol_type=“unassigned DNA” /note=“Right HsInv790_MLPA_REF probe” /organism=“Artificial Sequence”
caatccgtagtcttttgtccctcacctctagattggatcttgctggcac

SEQ ID NO: 87 - DNA, 53 bp, Artificial Sequence
  source 1..53: /mol_type=“unassigned DNA” /note=“Right HsInv832_MLPA_COM probe” /organism=“Artificial Sequence”
ctgtgtgatggaagaaggaaacagaagaggtctagattggatcttgctggcac

SEQ ID NO: 88 - DNA, 23 bp, Artificial Sequence
  source 1..23: /mol_type=“unassigned DNA” /note=“Reverse primer” /organism=“Artificial Sequence”
gtgccagcaagatccaatctaga

SEQ ID NO: 89 - DNA, 19 bp, Artificial Sequence
  source 1..19: /mol_type=“unassigned DNA” /note=“Forward primer labeled with FAM fluorocrom” /organism=“Artificial Sequence”
gggttccctaagggttgga

SEQ ID NO: 90 - DNA, 19 bp, Artificial Sequence
  source 1..19: /mol_type=“unassigned DNA” /note=“Forward primer labeled with VIC fluorocrom” /organism=“Artificial Sequence”
gggaaccgtagcacatgga

SEQ ID NO: 91 - DNA, 19 bp, Artificial Sequence
  source 1..19: /mol_type=“unassigned DNA” /note=“Forward primer labelled with NED fluorocrom” /organism=“Artificial Sequence”
gggtagggaatcccttgga

(19) United States
(12) Patent Application Publication
(10) Pub. No.: US 2015/0307918 A1
(43) Pub. Date: Oct. 29, 2015

(54) INVERSE MULTIPLEX LIGATION-DEPENDENT PROBE AMPLIFICATION (iMLPA), AN IN VITRO METHOD OF GENOTYPING MULTIPLE INVERSIONS

(71) Applicants: UNIVERSITAT AUTMÒNOMA DE BARCELONA, Bellaterra - Barcelona (ES); INSTITUCIÓ CATALANA DE RECERCA I ESTUDIS AVAÇANTS, Barcelona (ES)

(72) Inventors: Mario CÁCERES AGUILAR, Sant Cugat del Vallés (ES); Sergio VILLATORO GÓMEZ, Sabadell (ES); Cristina AGUADO ESTEBAN, Mollet del Vallés (ES)

(73) Assignees: UNIVERSITAT AUTÒNOMA DE BARCELONA, Bellaterra, Barcelona (ES); INSTITUCIÓ CATALANA DE RECERCA I ESTUDIS AVANÇATS, Barcelona (ES)

(21) Appl. No.: 14/647,718

(22) PCT Filed: Jul. 23, 2014
(86) PCT No.: PCT/EP2014/065841

§ 371 Date: May 27, 2015

(30) Foreign Application Priority Data
Jul. 23, 2013 (EP) 13382296.5

(51) Int. Cl. C12Q 1/68

(52) U.S. Cl. CPC C12Q 1/686

(57) ABSTRACT

It is described here a new method for improvement genotyping of a large number of inversions mediated by inverted repeats through a fast and high-throughput assay. The assay is based on Multiplex Ligation-dependent Probe Amplification, adapted for the detection of genomic structural variants, particularly adapted to inversions detection (iMLPA). By comparison with other techniques used to genotype inversions one by one, like inverse PCR, iMLPA has shown a very high sensibility, reproducibility and accuracy. Besides, iMLPA is the fastest method to determine the inversion genotypes in large sets of samples.

### FIELD OF THE INVENTION

This patent specification relates to the technical field of biomedicine. More specifically the patent discloses a new in vitro method, Inverse Multiplex Ligation-dependent Probe Amplification (iMLPA) for the detection of genomic inversions, one of the genetic structural variants existing in human genome.

### STATE OF THE ART

Within the field of biomedicine, there is a great interest to identify all genetic variants in humans and its association with phenotypic characteristics, including the susceptibility to different genetic diseases. Traditionally, the most studied genetic variants have been the changes in one nucleotide, known as single nucleotide polymorphisms or SNPs. During the last years, one of the major scientific breakthroughs has been the discovery of many other types of changes that affect bigger regions of the DNA, known as structural variants. Inversions are one class of structural variant that changes the orientation of one segment of the genome, usually without the insertion or deletion of DNA. However, inversions have been very little studied in humans due to the difficulty to determine if any individual carries a particular inversion or not.

The most traditional strategy for the analysis of large inversions is the standard G-banding karyotyping [1] and FISH [2-4]. Submicroscopic inversions have been detected using other techniques, like Southern or pulse-field gel electrophoresis (PFGE) [5,6]. The main problem is that none of these methods serves to study multiple inversions in a high number of individuals. Polymerase chain reaction (PCR) amplification offers more possibilities for high-throughput analysis and different PCR-based techniques have been used to validate inversions, including regular or long range PCR [7-11], haplotype-fusion PCR [12] or inverse PCR (iPCR) [13]. Regular or long-range PCR are limited by the size of the fragments to amplify and work poorly for fragments above 10 kb. Therefore, their applicability is reduced to inversions generated by simple breaks or small inverted repeats at their breakpoints. Haplotype-fusion PCR is a very promising technique to study inversions caused by duplicated sequences of almost any kind [12,14], although it has not been used yet extensively and reproducibly to genotype inversions. Inverse PCR [15] is based on creating circular molecules of DNA by restriction enzyme digestion and self-ligation of the two ends of the molecule, followed by amplification across the self-ligated ends with primers flanking a known restriction site. That way there is no need to amplify across the breakpoints and it is possible to analyze inversions mediated by medium-long inverted repetitive sequences. In particular, the iPCR has been used extensively to sequence the flanking regions of known sequences [16], sequence breakpoints of translocations [17,18], or generate long inserts pairs [19]. In addition, an iPCR assay has been developed to genotype inversions mediated by 9.5 kb segmental duplications causing hemophilia A in patients [13,20]. In this case, the circular molecules are between 12 kb and 21.6 kb and the protocol has been applied to multiple individuals in different studies [20-22] and in prenatal diagnosis [23]. However, all PCR techniques have the limitation that they are applied in a single-inversion basis and each inversion had to be assayed independently.

On the other hand, the multiplex ligation MLPA is a technique developed to overcome the limitations of multiplex PCR, WO2001/61033 A2 (SCHOUTEN, J. P.) 15 Feb. 2001 [24]. MLPA allows the relative quantification of several DNA fragments at the same time. Specifically, it has been used to study the copy number variation in specific regions of the genome and estimate the number of copies in each individual [25-27]. In addition, it has had a variety of other applications, such as the detection of mutations and SNPs [28], analysis of DNA methylation [29], or relative mRNA quantification [30], and it has been also applied to prenatal diagnosis of aneuploidies [31]. However, the MLPA method had never been used for the genotyping of inversions before.

The iMLPA method of present invention disclosed herein solves the problems still existing in the state of the art when facing detection of genomic structural variants by allowing multiple detection of genomic inversions in a simultaneous way, and by assaying at the same time a multiplicity of DNA samples. Moreover, due to the circularization by self-ligation that takes places in the iMLPA method, simultaneous detection of genomic regions which are not located adjacently in the same chromosome, is also feasible. Finally, the iMLPA method has the advantage that it requires a small quantity of DNA sample for genotyping multiple inversions at the same time.

### DESCRIPTION OF THE INVENTION

### BRIEF DESCRIPTION OF THE INVENTION

The technique of inverse MLPA (iMLPA) for the study of genomic inversions arises from the necessity to genotype or to detect, multiple inversions in a single assay in a quick and high-throughput manner. The main idea is to interrogate simultaneously as many inversions as possible in one sample and be able to analyze many samples in parallel. This opens the possibility to characterize in one experiment the frequency of these inversions in a group or population of interest. In particular, this technique is especially useful for inversions flanked by large repetitive sequences (<70 kb), which are precisely the ones most difficult to study by other methods. Therefore, the iMLPA would provide knowledge on the presence of all the inversions analyzed in any particular individual (personal genetic information). In addition, it is likely that in the near future associations between inversions and phenotypic traits or genetic diseases could be found, and the genotyping of inversions in an efficient way could have a more practical application (genetic testing).

The invention solves the technical problem existing in the state of the art of genotyping multiple inversions flanked by inverted repeats in many individuals at the same time.

The main innovative aspects of this technique, iMLPA, is the unforeseen: i) application of the MLPA technique to genotype inversions and, ii) the previous circularization by self-ligation of DNA fragments to join together sequences located originally far away and the application of the MLPA directly over this boundary. For that purposes the iMLPA protocol of the invention preferably works with restriction enzymes that generate staggered ends, in order to produce DNA fragments of a size that can be efficiently recircularized (so far <70 kb). It results then in a new and unexpected high-throughput assay to genotype or to detect multiple inversions.

In addition, in order to create a reliable and efficient assay, the development of the iMLPA went through an extensive process of improvement that affected many of its steps. This included:
(1) The design of the iMLPA probes and the adjustment of the amount of the probes in the mix to identify each of the orientations of all the inversions.
(2) Simplification of the process to increase the speed and the number of samples that can be analyzed by doing the restriction digestion and the circularization by self-ligation consecutively, without any purification step in between.
(3) Calculation of the amount of DNA (ranging 50-1000 ng per sample) and DNA dilution in order to maximize the efficiency of the self-ligation and the final PCR amplification.
(4) Development of the process of random DNA breakage and purification of the self-ligated fragments before the probe hybridization.

The term “primer”, as used herein, refers to an oligonucleotide of defined sequence that is designed to hybridize with a complementary, primer-specific portion of a target polynucleotide sequence and undergo primer extension. The primer can function as the starting point for the enzymatic polymerization of nucleotides. The primer should be long enough to prevent annealing to sequences other than the complementary portion. Generally, the primer is between 10 to 50 nucleotides in length. Preferably, the primer is between 13 to 30 nucleotides in length.

The term “probe”, as used herein, refers to an oligonucleotide that is capable of forming a duplex structure by complementary base pairing with a sequence of a target polynucleotide and is generally not able to form primer extension products.

For the purpose of present specification the term “comprises” or “comprising” means that, apart from the elements, ingredients or steps, specifically cited, the samples, assays, methods, may include, optionally, another elements, ingredients or steps, non-cited specifically. Also for purposes concerning present specification the term “comprises” or “comprising” includes terms such “consists” or “consisting”, limited to the cited elements, ingredients or steps.

Also for the purposes of present specification the term “genotyping” should be interpreted as detecting the status of genomic structural variants as, a way of example, genomic inversions, but also the reference standard normal orientation. More generally speaking, the term genotyping might be interpreted as the process of determining differences in the genetic make-up (genotype) of an individual by examining the individual's DNA sequence using biological assays and comparing it to another individual's sequence or a reference sequence.

As used herein, the term “nucleic acid” refers to a deoxyribonucleotide or ribonucleotide polymer, i.e. a polynucleotide, in either single-or double-stranded form, and unless otherwise limited, encompasses known analogues having the essential nature of natural nucleotides in that they hybridize to single-stranded nucleic acids in a manner similar to naturally occurring nucleotides (e. g., peptide encoding nucleic acids). Unless otherwise indicated, a particular nucleic acid sequence of the presently disclosed subject matter optionally comprises DNA as nucleic acid.

As used herein, the terms “restriction enzymes” refer to bacterial enzymes, each of which cut double-stranded DNA at or near a specific nucleotide sequence. Preferred restriction enzymes disclosed in the present specification are selected from: EcoRI, HindIII, SacI, NsiI, BamHI and BglII, or combinations thereof.

As used herein, the term “ligase” refers to a class of enzymes and their functions in forming a phosphodiester bond in adjacent oligonucleotides which are annealed to the same oligonucleotide. Particularly efficient ligation takes place when the terminal phosphate of one oligonucleotide and the terminal hydroxyl group of an adjacent second oligonucleotide are annealed together across from their complementary sequences within a double helix, i.e. where the ligation process ligates a “nick” at a ligatable nick site and creates a complementary duplex. The term “circularization by self-ligation or self-circularization” refers to the reaction of covalently joining the two ends of a DNA molecule through formation of an internucleotide linkage, creating a circular molecule. Ligases include DNA ligases and RNA ligases. A DNA ligase is an enzyme that closes nicks or discontinuities in one or both strands of duplex nucleic acids by creating an ester bond between juxtaposed 3′ OH and 5′ PO4 termini. DNA ligases include, but are not limited to, T4 DNA ligase, Taq DNA ligase, DNA ligase (E. coli) and the like. An RNA ligase is an enzyme that catalyzes ligation of juxtaposed 3′ OH and 5′ PO4 termini by the formation of a phosphodiester bond. RNA ligases include T4 RNA ligase 1, T4 ligase 2, TS2126 RNA ligase 1 and the like. A variety of ligases are commercially available (e.g., New England Biolabs, Beverly, Mass.).

Reference conformation, order or orientation should be defined in present specification as the normal or standard orientation actually present in the human reference genome sequence.

Therefore, present specification discloses herein an inverse multiplex ligation-dependent probe amplification (iMLPA) in vitro method for detecting in a sample, comprising a plurality of nucleic acids of different sequence, the presence of at least one specific genomic inversion structural variant, characterized by comprising, at least, the following successive steps:
i. Digesting nucleic acids comprised in the sample with restriction enzymes
ii. Circularization by self-ligation of the digested nucleic acid fragments with ligase enzymes
iii. Breaking nucleic acids obtained in the previous step (ii) and recovery of nucleic acids by purification
iv. Mixing recovered nucleic acids of previous step (iii) with a plurality of different probe pairs, each probe pair comprising:
   a. A first left nucleic acid oligonucleotide having a first target region complementary to one of the adjacent sequences of the circularized by self-ligation nucleic acid, which could be specific of the reference or inverted orientation or common for both orientations.
   b. A second right nucleic acid oligonucleotide having a second target region complementary to one of the adjacent sequences of the circularized by self-ligation nucleic acid, which could be specific of the reference or inverted orientation or common for both orientations.
v. Incubating the plurality of sample nucleic acids with the probe oligonucleotides allowing hybridization of complementary nucleic acids and ligation of the two parts of a probe pair that are complementary to the target sequence to form the final assembled probe.
vi. Amplifying the assembled probes by multiplex PCR, using at least 3 different pairs of universal labeled primers, wherein each pair of primers is formed by a common reverse primer and a specific forward primer in each case labeled with a different labeling compound.
vii. Detecting the amplicon or PCR amplification product.

Therefore, in a first aspect, the invention relates to an in vitro method for detecting the orientation of a genomic sequence within a larger sequence, wherein said genomic sequence is connected to the larger sequence at its 5′ and 3′ ends by a 5′ junction region and by a 3′ junction region in a sample comprising nucleic acids, said method comprising the following steps:
(i) digesting nucleic acids with at least a restriction enzyme, said restriction enzyme having at least a target site in the genomic sequence flanked by a junction region and at least another target site outside the genomic sequence flanked by a junction region,
(ii) circularizing the digested nucleic acid fragments obtained in step (i) by self-ligation with a ligase enzyme, thereby generating a circular nucleic acid comprising a junction region and a reconstituted target site for the restriction enzyme used in step (i), said reconstituted target site is flanked on one side by the region originally located 3′ with respect to the junction region and on the other side by the region originally located 5′ with respect to the junction region,
(iii) incubating the circularized nucleic acids obtained in step (ii) with at least a probe pair, each probe pair selected from the group consisting of:
   I. a probe pair comprising:
      a) a first oligonucleotide having a 5′ region and a 3′ region, wherein the 3′ region of said first oligonucleotide is complementary to a region of the genomic sequence flanked by a junction region and wherein the 3′ end of said first oligonucleotide is phosphorylated and
      b) a second oligonucleotide having a 5′ region and a 3′ region, wherein the 5′ region of said second oligonucleotide is complementary to a region of the larger sequence originally located outside the genomic sequence flanked by a junction region
   and wherein the nucleotide position within the circularized genomic sequence to which the 3′ end of the first oligonucleotide hybridizes and the nucleotide position within the genomic sequence to which the 5′ end of the second oligonucleotide hybridizes are adjacent positions, and
   wherein the region of the circularized genomic sequence to which the first and second oligonucleotide hybridize comprises the target site generated after the ligation step (ii), and
   II. a probe pair comprising:
      a) a first oligonucleotide having a 5′ region and a 3′ region, wherein the 3′ region of said first oligonucleotide is complementary to a region of the genomic sequence originally located outside the genomic sequence flanked by a junction region and wherein the 3′ end of said first oligonucleotide is phosphorylated and
      b) a second oligonucleotide having a 5′ region and a 3′ region, wherein the 5′ region of said second oligonucleotide is complementary to a region of the genomic sequence flanked by a junction region
      and wherein the nucleotide position within the circularized genomic sequence to which the 3′ end of the first oligonucleotide hybridizes and the nucleotide position within the genomic sequence to which the 5′ end of the second oligonucleotide hybridizes are adjacent positions, and
      wherein the region of the circularized genomic sequence to which the first and second oligonucleotide hybridize comprises the target site generated after the ligation step (ii),
(iv) ligating the 3′ end of the first oligonucleotide with the 5′ end of the second oligonucleotide of each probe pair to form an assembled probe,
(v) amplifying the assembled probe obtained in step (iv) by using a pair of primers, wherein the forward primer hybridizes to the 5′ region of the first oligonucleotide of the probe pair and the reverse primer hybridizes to the 3′ region of the second oligonucleotide of the probe pair, and
(vi) detecting the product of step (v).

The term “junction region”, as used herein, refers to a region that connects the genomic sequence which orientation is to be analyzed (i.e. the possible inversion) to the larger sequence of nucleic acid that contains said inversion. The junction region may be formed by a variable number of nucleotides. In an embodiment, the junction region is one nucleotide. In a preferred embodiment, the junction region is an inverted repeat.

In an embodiment, the restriction enzyme target site outside of the genomic sequence flanked by a junction region is located in a junction region. In another embodiment, the restriction enzyme target site outside of the genomic sequence flanked by a junction region is located outside of the junction region. In a preferred embodiment, the 5′ junction region and/or the 3′ junction region is an inverted repeat sequence. In a more preferred embodiment, if the 5′ junction region and the 3′ junction region are inverted repeat sequences, both are the same inverted repeat sequence. In a preferred embodiment, each inverted repeat sequence has up to 70 kb.

In a preferred embodiment, after step (ii) the nucleic acids are broken and recovered by purification.

In a preferred embodiment, the ligase enzyme used in step (ii) is T4 DNA ligase.

For detecting the amplicon or PCR amplification product, methods of standard MLPA are used [24].

iMLPA probes consist of two separate oligonucleotides, each containing one of the PCR primer sequences. The two probe oligonucleotides hybridize to immediately adjacent target sequences in the self-ligated molecules. Only when the two probe oligonucleotides are both hybridised to their adjacent targets can they be ligated during the ligation reaction. Because only ligated probes will be exponentially amplified during the subsequent PCR reaction, the number of probe ligation products is a measure for the number of target sequences in the sample. The size of the probe ligation products, combined with the specific label of the primer used in the PCR reaction, allows the identification of the target sequences present in the sample.

In a preferred embodiment, a plurality of different probe pairs is used wherein the 5′ region of the first oligonucleotide of each probe pair contains a nucleotide sequence of different length between the sequence complementary to the forward primer used in step (v) and the 3′ region of the first oligonucleotide. In another preferred embodiment, a plurality of different probe pairs is used wherein the 3′ region of the second oligonucleotide of each probe pair contains a nucleotide sequence of different length between the sequence complementary to the reverse primer used in step (v) and the 5′ region of the second oligonucleotide.

In an embodiment, the adjacent positions to which the 3′ end of the first oligonucleotide and the 5′ end of the second oligonucleotide hybridize are comprised within the target site generated after the ligation step (ii).

In a preferred embodiment, the probe pair is selected from the group consisting of SEQ ID NO: 1 to SEQ ID NO: 87 or combinations thereof. In a more preferred embodiment, the first oligonucleotide of the probe pair is selected from the group consisting of SEQ ID NO: 1 to SEQ ID NO: 48 or combinations thereof; and the second oligonucleotide of the probe pair is selected from the group consisting of SEQ ID NO: 49 to SEQ ID NO: 87 or combinations thereof.

In an embodiment, the ligase enzyme used in step (iv) is a NAD-dependent ligase enzyme. Preferably, is the ligase 65.

In an embodiment, the forward primer is labeled and when a plurality of pairs of primers is used in step (v), the forward primer of each pair is labeled with a different compound.

In another embodiment, the reverse primer is labeled and when a plurality of pairs of primers is used in step (v), the reverse primer of each pair is labeled with a different compound.

In a preferred embodiment, the labeling compound is selected from the group consisting of FAM, VIC, HEX/PET, TAMRA and NED.

In a preferred embodiment, the pair of primers used in step (v) is selected from the group consisting of SEQ ID NO: 88 and SEQ ID NO: 89; SEQ ID NO: 88 and SEQ ID NO: 90; SEQ ID NO: 88 and SEQ ID NO: 91, being SEQ ID NO: 88 the reverse primer and each of SEQ ID NO: 89, SEQ ID NO: 90 or SEQ ID NO: 91 the forward primer.

Particularly the iMLPA in vitro method is applied to samples comprising DNA as nucleic acid.

With the iMLPA in vitro method disclosed herein, at least 24 genomic inversions are detected simultaneously. More preferably, the said in vitro method detects inversions which are flanked by repetitive sequences having up to 70 kb, and preferably up to 50 kb.

Preferred restriction enzymes to be used according to the iMLPA in vitro method of invention are selected among those restriction enzymes which generate staggered ends. More preferred restriction enzymes are selected from: EcoRI, HindIII, SacI, NsiI, BamHI and BglII, or combinations thereof.

The most preferred ligase enzyme to be used in the iMLPA in vitro method of present invention is T4 DNA Ligase.

In the iMLPA in vitro method as disclosed herein, the probes, additionally to the target region of the sequence hybridizing specifically with their corresponding complementary parts of the DNA samples, also comprise a variable stuffer segment to adjust the probes lengths and still another sequence complementary to the forward or reverse universal primers used in multiplex PCR amplification.

For use in the iMLPA in vitro method of invention the probe pairs are selected from: SEQ ID No. 1 to SEQ ID No. 87 or combinations thereof.

In a preferred embodiment of the iMLPA in vitro method, the left probe is selected from: SEQ ID No: 1 to SEQ ID No: 48 or combinations thereof; and the right probe is selected from: SEQ ID No: 49 to SEQ ID No: 87, or combinations thereof.

Moreover, also for use in the iMLPA in vitro method as described herein, the pairs of universal primers are selected from: SEQ ID No. 88 and SEQ ID No. 89; SEQ ID No. 88 and SEQ ID No. 90; SEQ ID No. 88 and SEQ ID No. 91, being SEQ ID No. 88 the common reverse primer and each of SEQ ID No. 89, SEQ ID No. 90 or SEQ ID No. 91, specific forward primers, differentially labeled one from each other by a different fluorocrom. Specifically SEQ ID No. 89 was labeled with 6-carboxyfluorescein (FAM); SEQ ID No. 90 was labeled with VIC and SEQ ID No. 91 was labeled with NED.

The term, “fluorophore,” or “fluorocrom” as used herein refers to a species of excited energy acceptors capable of generating fluorescence when excited.

Part of present invention is also represented by the nucleic acid probes themselves, selected from any of SEQ ID No. 1 to SEQ ID No. 87 or by mixtures of nucleic acids comprising two or more probes selected from any of SEQ ID No. 1 to SEQ ID No. 87.

Therefore, in a second aspect, the invention relates to an oligonucleotide probe selected from the group consisting of any of SEQ ID NO: 1 to SEQ ID NO: 87 or mixtures thereof.

Present invention also concerns nucleic acid probes selected from any of SEQ ID No. 1 to SEQ ID No. 87 or mixtures of nucleic acids probes selected from any of SEQ ID No. 1 to SEQ ID No. 87, for use in the iMLPA in vitro method for detecting gene inversions detailed previously.

Finally the invention also comprises a kit for performing the iMLPA in vitro method previously detailed, the aforesaid kit comprising a nucleic acid probe selected from any of SEQ ID No. 1 to SEQ ID No. 87 or a mixture of probes selected from any of SEQ ID No. 1 to SEQ ID No. 87.

Therefore, in a third aspect, the invention relates to a kit comprising an oligonucleotide probe pair, wherein the first oligonucleotide of the probe pair is selected from the group consisting of SEQ ID NO: 1 to SEQ ID NO: 48 or combinations thereof; and the second oligonucleotide of the probe pair is selected from the group consisting of SEQ ID NO: 49 to SEQ ID NO: 87 or combinations thereof.

In a preferred embodiment, the kit further comprises a pair of primers selected from the group consisting of SEQ ID NO: 88 and SEQ ID NO: 89; SEQ ID NO: 88 and SEQ ID NO: 90; SEQ ID NO: 88 and SEQ ID NO: 91, being SEQ ID NO: 88 the reverse primer and each of SEQ ID NO: 89, SEQ ID NO: 90 or SEQ ID NO: 91 the forward primer.

In a more preferred embodiment, the forward primer or the reverse primer is labeled with a labeling compound. More preferably, the labeling compound is selected from the group consisting of FAM, VIC, HEX/PET, TAMRA and NED.

In another embodiment, the kit further comprises at least a reagent selected from the group consisting of:

a) a restriction enzyme and

b) a ligase enzyme

In a preferred embodiment, the restriction enzyme is selected from the group consisting of EcoRI, HindIII, SacI, NsiI, BamHI and BglII or combinations thereof.

In a preferred embodiment, the ligase enzyme is selected from the group consisting of T4 DNA ligase and a NAD-dependent ligase enzyme.

As used herein, the term “kit” refers generally to a collection of containers containing the necessary elements to carry out the process of the invention in an arrangement both convenient to the user and which maximizes the chemical stability of the elements. Such a kit may comprise a carrier being compartmentalized to receive in close confinement therein one or more containers, such as tubes or vials, as well as printed instructions including a description of the most preferred protocols for carrying out the methods of the invention in a particular application. As used herein, the term “kit” refers to any delivery system for delivering materials. In the context of reaction assays, such delivery systems include systems that allow for the storage, transport, or delivery of reaction reagents (e.g., oligonucleotides, enzymes, probes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials.

### FIGURES DESCRIPTION

FIG. 1. Process of DNA preparation and probe hybridization for the iMLPA assay. Reference and inverted conformation, order or orientation are represented by unique regions A, B, C and D, which are separated by the inverted repeats IR1 and IR2 at each inversion breakpoint (BP). The iMLPA involves four main steps: restriction enzyme digestion at the target sites (RE), circularization by self-ligation of the fragments produced by digestion, hybridization of the iMLPA probes to interrogate specifically each DNA orientation for inversion genotyping followed by ligation of the adjacent probes, and multiplex PCR amplification of the ligated or assembled probes.

FIG. 2. Diagram showing the main steps of the iMLPA probe hybridization and amplification. 1. Hybridization of the iMLPA probe oligonucleotides to adjacent sites created by the circularization of the DNA molecule of interest. 2. Ligation of the 2 adjacent probe oligonucleotides (marked by an arrow) to form the assembled probe. 3. Multiplex PCR amplification of the ligated or assembled probes.

### DETAILED DESCRIPTION OF THE INVENTION

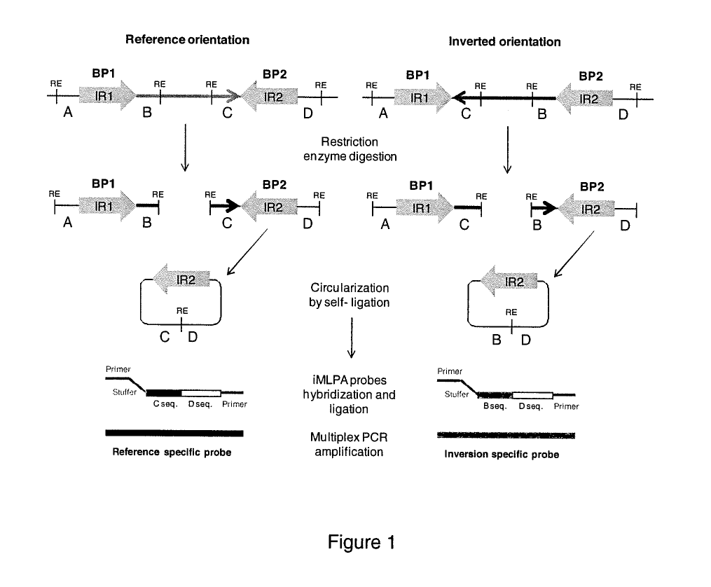

The iMLPA technique is based on the custom MLPA assay, which uses specific probes designed precisely to study a region of interest, with unexpected and important changes and improvements in the previous treatment of DNA samples to be analyzed. At the experimental level it includes four main steps (FIG. 1) and all the successive reactions are carried out in a 96-well plate format to maximize speed and throughput. Those 4 steps are detailed in the following examples 1-4.

### EXAMPLE 1

### Digestion of DNA with Restriction Enzymes

For the preparation of the samples for iMLPA, first we selected a concentration of genomic DNA between 300-800 ng of each individual. In the present example, 400 ng of genomic DNA of each individual are digested overnight at 37° C. under conditions recommended by the manufacturer in a 20 μl reaction with 5 U of the appropriate restriction enzyme. In our case we used the restriction enzymes EcoRI, HindIII, SacI, BamHI from Roche and NsiI and BglII from New England Biolabs. The restriction enzymes are then inactivated at 65° C. for 15 minutes, with the exception of BglII that is inactivated at 85° C. for 20 minutes.

### EXAMPLE 2

### Self-Ligation of the Digested Fragments

In the second step, circularization by self-ligation of the DNA fragments is performed for 16 hours at 16° C. in an incubator by mixing the 20 μl of the digestion reaction of each enzyme (totaling 120 μl) in a total volume of 640 μl with 400 U of T4 DNA Ligase (New England Biolabs), 64 μl of the ligation buffer provided by the manufacturer, and 455 μl of water. This results in a concentration of the DNA fragments generated by each enzyme of 0.625 ng/μl, which is optimal for self-ligation and subsequent processes. Next, in one step, the ligation is inactivated and the DNA is broken at 95° C. for 5 min in order to make its recovery easier. Finally the DNA is put in ice for at least 5 minutes.

### EXAMPLE 3

### DNA Recovery

The DNA recovery is carried out using the kit ZR-96 DNA Clean & Concentrator™-5 (Zymo Research) according to the instructions provided by manufacturer. Briefly, two volumes (1280 μl) of DNA Binding Buffer are added to the ligation volume, vortexed for 30 sec, and left at least 5 min at room temperature. The mixture is then loaded into a Zymo-Spin™ I-96 Plate and centrifuged. Next, 300 μl of DNA Wash Buffer were added to each well and centrifuged, and the washing step is repeated two times. DNA from each sample is finally resuspended by adding 12 μl of water, obtaining at the end approximately 7.5 μl of recovered DNA.

### EXAMPLE 4

### Detection of Inversions

For the detection of each of the inversions, two iMLPA probe pairs are used to interrogate the two orientations, either the reference or the inverted. The iMLPA probes are specifically designed using the program Proseek [32] and manually modified to hybridize around the restriction enzyme target sequences, where the self-ligation of the DNA is expected to have occurred. At this position, one probe of the probe pair is located within the inverted region and the other probe of the probe pair is outside (FIG. 1), and it is possible to interrogate the orientation of the DNA molecule from which the DNA fragment was originated. Specifically, each iMLPA probe pair is formed by two oligonucleotides that target adjacent sequences in the self-ligated DNA, in which both oligonucleotides might be specific of the reference or inverted orientation or common for the two orientations (FIG. 1). Besides the sequence specific to its target, each probe oligonucleotide has a variable stuffer segment to adjust the length of the final assembled probes, and a sequence complementary to the forward or reverse universal primers for multiplex PCR amplification of the complete probes. Taking advantage of the high specificity of the MLPA technique, so far we have designed 48 different custom iMLPA probe pairs formed by 87 different probe oligonucleotide sequences and mixed them in a single mix (iMLPA MIX) in order to score the genotypes of 24 different inversions (Table 1 and 2).

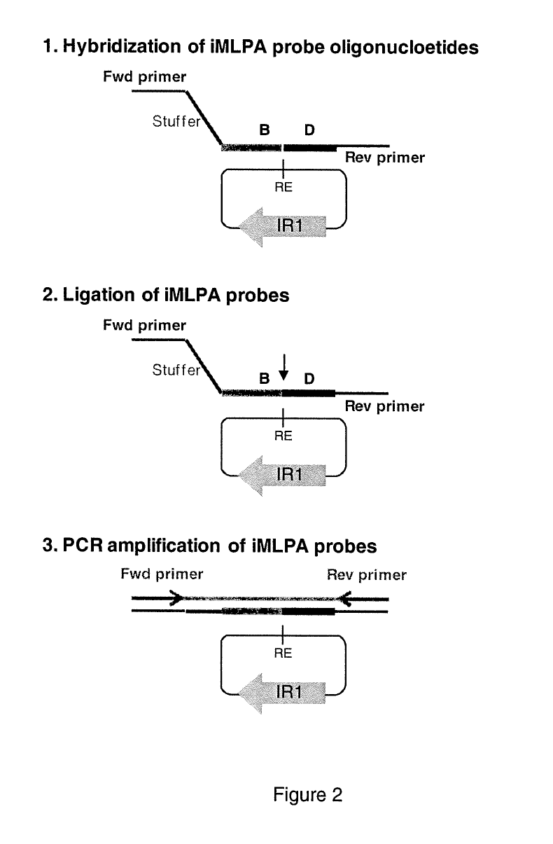

The last step is to perform the regular MLPA assay following the manufacturer instructions with only minor modifications (FIG. 2). For each sample, the 7.5 μl of the recovered DNA is heated at 98° C. for 90 sec to complete the fragmentation of DNA. Then, the temperature is reduced to 25° C. and 1.5 μl of our iMLPA MIX of probes and 1.5 μl of Salsa MLPA buffer (MRC-Holland) are added. In order to denature the DNA and iMLPA MIX probes simultaneously, the temperature is raised again up to 95° C. for 90 sec and decreased to 60° C. for 16 hours to ensure the correct hybridization of the probes. Next, the ligation of adjacent probes is performed at 54° C. for 25 min by adding 25 μl of water and 1 μl of Ligase 65, 3 μl of Salsa buffer A and 3 μl of Salsa buffer B (MRC-Holland). After this, ligation is inactivated at 95° C. for 5 min and PCR is performed separately for groups of 8-9 inversions using three different pairs of universals primers previously described [27]. These universal primer pairs are formed by a common reverse primer (GTGCCAGCAAGATCCAATCTAGA) (SEQ ID No. 88) and a specific forward primer in each case labeled with a different fluorocrom: FAM, GGGTTCCCTAAGGGTTGGA (SEQ ID No. 89); VIC, GGGAACCGTAGCACATGGA (SEQ ID No. 90); and NED, GGGTAGGGAATCCCTTGGA (SEQ ID No. 91). In each PCR reaction, 6 μl of the iMLPA hybridization-ligation template are added in a volume of 25 μl, containing 2 μl of Salsa PCR (MRC-Holland), 13.5 μl of water, 1 μM of dNTPs, 0.2 μM of the universal forward and reverse primers (forward primer labeled with FAM, VIC or NED), 1 μl of PCR buffer without MgCl₂ (Roche), and 2.5 U of Taq DNA polymerase (Roche). Amplification is carried out by an initial denaturation step of 15 sec at 95° C., followed by 47 cycles of 95° C. for 30 sec, 60° C. for 30 sec, and 72° C. for 60 sec, and a final extension at 72° C. for 25 min. Finally, 5 μl of the amplification products of the three PCR reactions labeled with FAM, VIC or NED are mixed and 2 μl of the mix are analyzed by capillary electrophoresis using an ABI PRISM 3130 Genetic Analyzer sequencer (Applied Biosystems). Each complete probe has a unique combination of length and fluorochrom label, so the peaks can be separated and visually inspected using the GeneScan version 3.7 software. That way it is possible to determine the genotypes for a total of 24 inversions in a single run.

TABLE 1
Set of iMLPA left probes used to genotype 24 polymorphic inversions in the human
genome. The table shows the Left iMLPA probe name, the restriction enzyme used
for the DNA digestion, their chromosomal location in the genome NCBI Build 36.1
(HG18) genome version, and the sequence of each oligonucleotide. Besides, the
amount of each oligonucleotide in a 1 μM concentration necessary to generate
enough iMLPA MIX for four 96-well plates by adding 48.2 μl of water (final
volume of 600 μl) is also specified.

| | | | Left | | SEQ | |
|---|---|---|---|---|---|---|
| | | | probe | | ID | MIX |
| Probe ID | Enzyme | Chr | location | Left iMLPA probe | No. | μl |
| HsInv030_MLPA | HindIII | 16 | 73803940- | GGGTAGGGAATCCCTTGGACCTTCCCCTTCCCTCCATGAA | 1 | 1.7 |
| _INV | | | 73803960 | | | |
| HsInv030_MLPA | HindIII | 16 | 73819800- | GGGTAGGGAATCCCTTGGAcattCAGGGGTTCCAAGCACCCTGAAG | 2 | 0.8 |
| _REF | | | 73819825 | | | |
| HsInv031_MLPA | EcoRI | 16 | 83746706- | GGGAACCGTAGCACATGGAccttgcGCTGGATCTTTGCTGGTGTTTTGCTC | 3 | 0.6 |
| _INV | | | 83746739 | ATGTATTG | | |
| HsInv031_MLPA | EcoRI | 16 | 83746672- | GGGAACCGTAGCACATGGAcctggagcgacctgtgagatagAACAAATTCT | 4 | 3.9 |
| _REF_2 | | | 83746701 | CTCCATGTTTG | | |
| HsInv040_MLPA | HindIII | 2 | 138726050- | GGGTAGGGAATCCCTTGGAcctatagcgacttacggacggcgtattcgtac | 5 | 14 |
| _INV | | | 138726072 | tgactgcccGGTCTTGAAAATGTTGCTTAAGC | | |
| HsInv040_MLPA | HindIII | 2 | 138722625- | GGGTAGGGAATCCCTTGGAcctccCCATTGACAAGAGAGTCAATTTGTCCT | 6 | 9.8 |
| _REF | | | 138722655 | CTGA | | |
| HsInv045_MLPA | SacI | 21 | 26943471- | GGGAACCGTAGCACATGGAcctatagcgactCCAGCCCCCTATGTGGGTTT | 7 | 14 |
| _INV_2 | | | 26943493 | CTA | | |
| HsInv045_MLPA | SacI | 21 | 26948167- | GGGAACCGTAGCACATGGAcctatagcgactGCATCCCACTTTTGGAATGC | 8 | 4 |
| _REF_2 | | | 26948201 | CATATTCTAGAGCTC | | |
| HsInv055_MLPA | BamHI | 5 | 63806260- | GGGAACCGTAGCACATGGActtCTTAGCAGAGCTCGAGCACTGTGCTGG | 9 | 7.2 |
| _INV | | | 63806292 | GGGATC | | |
| HsInv055_MLPA | BamHI | 5 | 63806315- | GGGAACCGTAGCACATGGAcctatagtCAGTCAGGAGGCATGAGGGTCAG | 10 | 4.8 |
| _INV_bis | | | 63806342 | GGATC | | |
| HsInv055_MLPA | BamHI | 5 | 63805808- | GGGAACCGTAGCACATGGAcctaaagccagggagccaagtggtcttgctca | 11 | 5 |
| _REF | | | 63805845 | gtggatc | | |
| HsInv061_MLPA | BglII | 6 | 107278575- | GGGTAGGGAATCCCTTGGAGACGTGTAGGGCTTGCAGGCATGGA | 12 | 0.8 |
| _INV | | | 107278599 | | | |
| HsInv061_MLPA | BglII | 6 | 107271731- | GGGTAGGGAATCCCTTGGAccatGAGGTGGTGGTTGCAGTGAGCCGAGA | 13 | 1.5 |
| _REF | | | 107271757 | T | | |
| HsInv072_MLPA | HindIII | X | 45437924- | GGGTAGGGAATCCCTTGGAcctatagcgacttacggacggcgtaccCCTTA | 14 | 11 |
| _INV | | | 45437947 | TGTGGGCTTACCGAAGCTT | | |
| HsInv072_MLPA | HindIII | X | 45433531- | GGGTAGGGAATCCCTTGGAcctatagcgacttacggacggcgtatccgacC | 15 | 12 |
| _REF | | | 45433575 | TGTATCCTGAGACTTTGCTGAAGTTGCTTATCAGCTTAAGAAGC | | |
| HsInv114_MLPA | BamHI | 9 | 126748269- | GGGAACCGTAGCACATGGAcctatagcgacttacggacggcgtatccgaCC | 16 | 1.5 |
| _INV_2 | | | 126748296 | TGACTTATGGAACGAATGAGTCAGTG | | |
| HsInv114_MLPA | BamHI | 9 | 126764219- | GGGAACCGTAGCACATGGAcctatagcgacttacggacggcgtatccgact | 17 | 2 |
| _REF_2 | | | 126764245 | ccttgcctCACATGCTCAAGACAACAACCCTTGG | | |
| HsInv124_MLPA | HindIII | 11 | 317060- | GGGTTCCCTAAGGGTTGGAcctataCTCTAGGGCCCCACTGGCCAAAAGC | 18 | 1 |
| _COM_2 | | | 317086 | TT | | |
| HsInv124_MLPA | HindIII | 11 | 317060- | GGGTTCCCTAAGGGTTGGAcctataCTCTAGGGCCCCACTGGCCAAAAGC | 18 | 1 |
| _COM_2 | | | 317086 | TT | | |
| HsInv209_MLPA | HindIII | 11 | 70965274- | GGGTTCCCTAAGGGTTGGAcctatagcgactatacatCATTCCCACAGGAA | 19 | 2 |
| _INV | | | 70965301 | TGTGCCAAGAGAAG | | |
| HsInv209_MLPA | HindIII | 11 | 70961694- | GGGTTCCCTAAGGGTTGGAcctatagcgactatacaCAAGGTTGCATCGTG | 20 | 2 |
| _REF | | | 70961725 | ACCACgggcctggaaag | | |
| HsInv278_MLPA | BglII | 5 | 180463471- | GGGTTCCCTAAGGGTTGGAcctatagcgacttacggacgacgtatacgctg | 21 | 2.4 |
| _INV | | | 180463492 | cctttgctcgcagatct | | |
| HsInv278_MLPA | BglII | 5 | 180459934- | GGGTTCCCTAAGGGTTGGAcctatagcgacttacggacggcgtaCATGGAT | 22 | 2.4 |
| _REF | | | 180459960 | GCAGCTCTTGTCCTAAGAGA | | |
| HsInv340_MLPA | BamHI | 13 | 63266920- | GGGTTCCCTAAGGGTTGGAcatcCATATCAGTTTTGGGTTGGAGGGATG | 23 | 16.8 |
| _INV_2 | | | 63266949 | | | |
| HsInv340_MLPA | BamHI | 13 | 63203502- | GGGTTCCCTAAGGGTTGGAcctatagcGGTAAGTATGACATTACATGTTTC | 24 | 7 |
| _REF | | | 63203533 | TTGGATCC | | |
| HsInv341_MLPA | NsiI | 13 | 79311179- | GGGTAGGGAATCCCTTGGAcctatagcgacttacggaccGGTTCCATGGTC | 25 | 2.6 |
| _INV | | | 79311210 | AAGAATTTGAAAAGAGATGC | | |
| HsInv341_MLPA | NsiI | 13 | 79301403- | GGGTAGGGAATCCCTTGGAcctatagcgacttacggacggcgtattatCAT | 26 | 2 |
| _REF | | | 79301428 | AGTGGCAGGGCAGGATGCTATGC | | |
| HsInv344_MLPA | HindIII | 14 | 34116164- | GGGTTCCCTAAGGGTTGGAcctatagcgacttacggacggaCTAGTAGCTG | 27 | 16.8 |
| _INV | | | 34116197 | GGATTACAGGTGCACGTCACCAAG | | |
| HsInv344_MLPA | HindIII | 14 | 34093428- | GGGTTCCCTAAGGGTTGGAcctaagcaCATGAGGGTCTTGTAGACACCACA | 28 | 9.6 |
| _REF_2 | | | 34093466 | GTAAAG | | |
| HsInv347_MLPA | EcoRI | 14 | 60145521- | GGGTAGGGAATCCCTTGGAcctatagcgacttacggacggcgcCCCATCAA | 29 | 12.2 |
| _INV | | | 60145550 | AAGAATAACTGCAGGGATGGGA | | |
| HsInv347_MLPA | EcoRI | 14 | 60145490- | GGGTAGGGAATCCCTTGGAcctatagcgacttacggacggcgtattgCGAG | 30 | 2.4 |
| _REF | | | 60145518 | GTGTTTCCCTCTTCCCTGATTATGA | | |
| HsInv374_MLPA | EcoRI | 17 | 25975205- | GGGAACCGTAGCACATGGAccgccGGCCTACTTACTTTGTATATAAATGT | 31 | 0.8 |
| _INV | | | 25975426 | GTAAACTCCTCAA | | |
| HsInv374_MLPA | EcoRI | 17 | 25975162- | GGGAACCGTAGCACATGGAccgccgtcggGACGTTGAACTAATTTCCTTAT | 32 | 0.8 |
| _REF | | | 25975198 | TGGAGTTCATTATTG | | |
| HsInv379_MLPA | BamHI | 19 | 22043254- | GGGAACCGTAGCACATGGAcCCTGCTGCAGTTACATGAGAGGATC | 33 | 1 |
| _INV | | | 22043278 | | | |
| HsInv379_MLPA | BamHI | 19 | 22043250- | GGGAACCGTAGCACATGGAcctGTGACCTGCTGCAGTTACATGAGAG | 34 | 0.5 |
| _REF | | | 22043274 | | | |
| HsInv389_MLPA | NsiI | X | 153264503- | GGGTTCCCTAAGGGTTGGAcCAGCCCTGCCTCCACAAATG | 35 | 1 |
| _INV | | | 153264522 | | | |
| HsInv389_MLPA | NsiI | X | 153229291- | GGGTTCCCTAAGGGTTGGACCTGGGATTGGCACCTTGAATG | 36 | 1 |
| _REF | | | 153229312 | | | |
| HsInv393_MLPA | BglII | X | 100760471- | GGGTTCCCTAAGGGTTGGAcctatagcgacttacggacggcCTGGCTGAAC | 37 | 4.8 |
| _INV | | | 100760508 | TCATAGTGTTAGGTGTCAGATGACTGAG | | |
| HsInv393_MLPA | BglII | X | 100745056- | GGGTTCCCTAAGGGTTGGAcctatagcgacttacggacggcgtattcgtca | 38 | 4.8 |
| _REF | | | 100745087 | GCATCTCACAAAGACCAATTGTCAATACGTAG | | |
| HsInv396_MLPA | EcoRI | 11 | 72229400- | GGGTAGGGAATCCCTTGGAcctatagcgacCGTTGAATTTGATTTTGGGTC | 39 | 16.2 |
| _INV | | | 72229428 | TCAGCCAC | | |
| HsInv396_MLPA | EcoRI | 11 | 72229400- | GGGTAGGGAATCCCTTGGAcctatagcgactatacaCGTTGAATTTGATTT | 40 | 12 |
| _REF | | | 72229428 | TGGGTCTCAGCCAC | | |
| HsInv397_MLPA | SacI | X | 105414000- | GGGAACCGTAGCACATGGAcctgtagcgacttaGAATTGGCTATGGGGAAA | 41 | 9.6 |
| _INV_2 | | | 105414028 | TAACTGAGCTC | | |
| HsInv397_MLPA | SacI | X | 105412636- | GGGAACCGTAGCACATGGAccttGATCTTGGATGAGGCCACCCTCAAGGC | 42 | 12.4 |
| _REF_2 | | | 105412677 | TGAGACCCAGAGCTC | | |
| HsInv403_MLPA | HindIII | X | 75283893- | GGGTAGGGAATCCCTTGGAcaccCTCCCTGTGGAGAGACTGTCGTCAGA | 43 | 8 |
| _INV | | | 75283947 | CCAACTCAAAATTACAAAGTTTTCCAAAG | | |
| HsInv403_MLPA | HindIII | X | 75292078- | GGGTAGGGAATCCCTTGGAcctatagcgacttacggacggcgtattcCTGC | 44 | 12 |
| _REF | | | 75292103 | ATTTCAGTGTTAAGGCCCAGAA | | |
| HsInv790_MLPA | BamHI | 17 | 18661875- | GGGAACCGTAGCACATGGAcctGGCAGACTGTCCAGATAGGAACCTTG | 45 | 6 |
| _INV | | | 18661900 | | | |
| HsInv790_MLPA | BamHI | 17 | 18480175- | GGGAACCGTAGCACATGGAcctatgaGGATCAGGCAAAGGGGAAATTGGA | 46 | 7 |
| _REF | | | 18480200 | TC | | |
| HsInv832_MLPA | BamHI | Y | 16511539- | GGGTAGGGAATCCCTTGGAcGACTTTTGTATCAGGTGTAAGGATGGGAT | 47 | 2.6 |
| _INV | | | 16511568 | C | | |
| HsInv832_MLPA | BamHI | Y | 16511510- | GGGTAGGGAATCCCTTGGAcG | 48 | 3 |
| _REF | | | 16511543 | GCTAGCCATATGTAGAAAGCT | | |
| | | | | GAAACTGGATC | | |

TABLE 2
Set of iMLPA right probes used to genotype 24 polymorphic inversions in the human
genome. The table shows the Right iMLPA probe name, the restriction enzyme used
for the DNA digestion, their chromosomal location in the genome NCBI Build 36.1
(HG18) genome version, and the sequence of each oligonucleotide. Besides, the
amount of each oligonucleotide in a 1 μM concentration necessary to generate
enough iMLPA MIX for four 96-well plates by adding 48.2 μl of water (final
volume of 600 μl) is also specified. According to the original MLPA strategy,
the right oligonucleotide is phosphorylated at its 5′ end to increase
specificity.

| | | | Right | | SEQ | |
|---|---|---|---|---|---|---|
| | | | probe | | ID | MIX |
| Probe ID | Enzyme | Chr | location | Right iMLPA probe | No. | μl |
| HsInv030_MLPA | HindIII | 16 | 73793321- | GCTTGCCTCCTGAAATACTTTTATGAGcTCTAGATTGGATCTTGCTG | 49 | 1.7 |
| _INV | | | 73793347 | GCAC | | |
| HsInv030_MLPA | HindIII | 16 | 73803939- | CTTCATGGAGGGAAGGGGAAGGCTCTCTAGATTGGATCTTGCTGGCA | 50 | 0.8 |
| _REF | | | 73803963 | C | | |
| HsInv031_MLPA | EcoRI | 16 | 83742839- | AATTCCCTCCTCCTGGGAGAGGTCTAGATTGGATCTTGCTGGCAC | 51 | 0.6 |
| _COM_2 | | | 83742860 | | | |
| HsInv031_MLPA | EcoRI | 16 | 83742839- | AATTCCCTCCTCCTGGGAGAGGTCTAGATTGGATCTTGCTGGCAC | 51 | 3.9 |
| _COM_2 | | | 83742860 | | | |
| HsInv040_MLPA | HindIII | 2 | 138722625- | TTCAGAGGACAAATTGACTCTCTTGTCAATGGCTCTAGATTGGATCT | 52 | 14 |
| _INV | | | 138722656 | TGCTGGCAC | | |
| HsInv040_MLPA | HindIII | 2 | 138717831- | AGCTTAATTTAATACTTACTTTTACTAGCTTATTATAAAGGATACAT | 53 | 9.8 |
| _REF | | | 138717890 | CTCAGGAACAGCGccccTCTAGATTGGATCTTGCTGGCAC | | |
| HsInv045_MLPA | SacI | 21 | 26926955- | GAGCTCTTCGTAAATTAGCCTGTCTAGAAATTCTCTAGATTGGATCT | 54 | 14 |
| _INV_2 | | | 26926987 | TGCTGGCAC | | |
| HsInv045_MLPA | SacI | 21 | 26943471- | TAGAAACCCACATAGGGGGCTGGGTCTAGATTGGATCTTGCTGGCAC | 55 | 4 |
| _REF_2 | | | 26943494 | | | |
| HsInv055_MLPA | BamHI | 5 | 63772352- | cagaggccagcccaagtggctgcctagttctcttagacTCTAGATTG | 56 | 7.2 |
| _COM | | | 63772389 | GATCTTGCTGGCAC | | |
| HsInv055_MLPA | BamHI | 5 | 63772352- | cagaggccagcccaagtggctgcctagttctcttagacTCTAGATTG | 56 | 5 |
| _COM | | | 63772389 | GATCTTGCTGGCAC | | |
| HsInv061_MLPA | BglII | 6 | 107277299- | AGATCTCGGCTCACTGCAACCACCACCTCCTCTAGATTGGATCTTGC | 57 | 0.8 |
| _INV | | | 107277327 | TGGCAC | | |
| HsInv061_MLPA | BglII | 6 | 107277299- | CTGTCTGAGGCCAAAGTCTACAACTTCTCTAGATTGGATCTTGCTGG | 58 | 1.5 |
| _REF | | | 107277325 | CAC | | |
| HsInv072_MLPA | HindIII | X | 45433520- | CTTAAGCTGATAAGCAACTTCAGCAAAGTCTCAGGATACAGAATCAA | 59 | 11 |
| _INV | | | 45433571 | CTGTGTCTAGATTGGATCTTGCTGGCAC | | |
| HsInv072_MLPA | HindIII | X | 45430544- | TTCTATGCCACAGAGGCAAATCAGCATTCCTCTAGATTGGATCTTGC | 60 | 12 |
| _REF | | | 45430573 | TGGCAC | | |
| HsInv114_MLPA | BamHI | 9 | 126732616- | GATCCTCTCAAGGGAGAGCCCAAGGCTGGTGTTCTCTAGATTGGATC | 61 | 1.5 |
| _INV_2 | | | 126732649 | TTGCTGGCAC | | |
| HsInv114_MLPA | BamHI | 9 | 126748265- | GATCCACTGACTCATTCGTTCCATAAGTCTCTAGATTGGATCTTGCT | 62 | 2 |
| _REF_2 | | | 126748293 | GGCAC | | |
| HsInv124_MLPA | HindIII | 11 | 302279- | CTTTAAATCACGGGCAGTTTAGGAAGGTCTAGATTGGATCTTGCTGG | 63 | 1 |
| _INV_ 2 | | | 302305 | CAC | | |
| HsInv124_MLPA | HindIII | 11 | 302312- | CCAAAATACCTTCCACGGGAAATTCAAGCcTCTAGATTGGATCTTGC | 64 | 1 |
| _REF | | | 302341 | TGGCAC | | |
| HsInv209_MLPA | HindIII | 11 | 70951461- | cttcccaggtgagctgagtcttatccTCTAGATTGGATCTTGCTGGC | 65 | 2 |
| _COM | | | 70951486 | AC | | |
| HsInv209_MLPA | HindIII | 11 | 70951461- | cttcccaggtgagctgagtcttatccTCTAGATTGGATCTTGCTGGC | 65 | 2 |
| _COM | | | 70951486 | AC | | |
| HsInv278_MLPA | BglII | 5 | 180459929- | CTTAGGACAAGAGCTGCATCCATGGACAGTCTAGATTGGATCTTGCT | 66 | 2.4 |
| _INV | | | 180459957 | GGCAC | | |
| HsInv278_MLPA | BglII | 5 | 180446114- | tcttgtcataaacacagatcccaggctgcTCTAGATTGGATCTTGCT | 67 | 2.4 |
| _REF | | | 180446142 | GGCAC | | |
| HsInv340_MLPA | BamHI | 13 | 63203497- | GATCCAAGAAACATGTAATGTCATACTTACCTAATCTCTAGATTGGA | 68 | 16.8 |
| _INV_2 | | | 63203532 | TCTTGCTGGCAC | | |
| HsInv340_MLPA | BamHI | 13 | 63171106- | TCATGCCTTCTAGTTTGTAGGGTTTCTGCTCTAGATTGGATCTTGCT | 69 | 7 |
| _REF | | | 63171134 | GGCAC | | |
| HsInv341_MLPA | NsiI | 13 | 79284287- | ATTCAGCCAGTCATTCATGATGTTCCCTCTAGATTGGATCTTGCTGG | 70 | 2.6 |
| _COM | | | 79284313 | CAC | | |
| HsInv341_MLPA | NsiI | 13 | 79284287- | ATTCAGCCAGTCATTCATGATGTTCCCTCTAGATTGGATCTTGCTGG | 70 | 2 |
| _COM | | | 79284313 | CAC | | |
| HsInv344_MLPA | HindIII | 14 | 34093434- | CTTTACTGTGGTGTCTACAAGACCCTCATGATCTCTAGATTGGATCT | 71 | 16.8 |
| _INV | | | 34093466 | TGCTGGCAC | | |
| HsInv344_MLPA | HindIII | 14 | 34077708- | CTTCTTTAGGCAGAATGAATGTTTTAAAGTTTAAGAATAGGATCTGC | 72 | 9.6 |
| _REF_2 | | | 34077761 | TGACAGCTCTAGATTGGATCTTGCTGGCAC | | |
| HsInv347_MLPA | EcoRI | 14 | 60136285- | ATTCTCTTTCAGGCATGTGATTTCATAGGACTCTAGATTGGATCTTG | 73 | 12.2 |
| _COM | | | 60136315 | CTGGCAC | | |
| HsInv347_MLPA | EcoRI | 14 | 60136285- | ATTCTCTTTCAGGCATGTGATTTCATAGGACTCTAGATTGGATCTTG | 73 | 2.4 |
| _COM | | | 60136315 | CTGGCAC | | |
| HsInv374_MLPA | EcoRI | 17 | 25966851- | GAATTCTAATATTACTCCTAAAGGGAAAAATCTATGGGcgccTCTAG | 74 | 0.8 |
| _COM | | | 25966888 | ATTGGATCTTGCTGGCAC | | |
| HsInv374_MLPA | EcoRI | 17 | 25966851- | GAATTCTAATATTACTCCTAAAGGGAAAAATCTATGGGcgccTCTAG | 74 | 0.8 |
| _COM | | | 25966888 | ATTGGATCTTGCTGGCAC | | |
| HsInv379_MLPA | BamHI | 19 | 21624227- | CCAAGCAAATCACAGCGGCCCTACTCTAGATTGGATCTTGCTGGCAC | 75 | 1 |
| _INV | | | 21624250 | | | |
| HsInv379_MLPA | BamHI | 19 | 22032114- | GATCCACAGGCAGATGCAGTTAAGGTCTAGATTGGATCTTGCTGGCA | 76 | 0.5 |
| _REF | | | 22032138 | C | | |
| HsInv389_MLPA | NsiI | X | 153217300- | CATGGAGGACAGGCGATGGGGTCTAACTCTAGATTGGATCTTGCTGG | 77 | 1 |
| _COM | | | 153217326 | CAC | | |
| HsInv389_MLPA | NsiI | X | 153217300- | CATGGAGGACAGGCGATGGGGTCTAACTCTAGATTGGATCTTGCTGG | 77 | 1 |
| _COM | | | 153217326 | CAC | | |
| HsInv393_MLPA | BglII | X | 100745056- | ATCTACGTATTGACAATTGGTCTTTGTGAGATGCTCTAGATTGGATC | 78 | 4.8 |
| _INV | | | 100745089 | TTGCTGGCAC | | |
| HsInv393_MLPA | BglII | X | 100737513- | ATCTGTGGGAAAGTCAAATCTTTTTGATCCAGCCTCTAGATTGGATC | 79 | 4.8 |
| _REF | | | 100737546 | TTGCTGGCAC | | |
| HsInv396_MLPA | EcoRI | 11 | 72144566- | GAATTCATATTCACAATAAATATTCCAAGACCccTCTAGATTGGATC | 80 | 16.2 |
| _INV | | | 72144597 | TTGCTGGCAC | | |
| HsInv396_MLPA | EcoRI | 11 | 72213808- | GAATTCAATAGAATATTAAGAGCCAGAGccTCTAGATTGGATCTTGC | 81 | 12 |
| _REF | | | 72213835 | TGGCAC | | |
| HsInv397_MLPA | SacI | X | 105393680- | aaaacacaaatccgttgaggttcagaatcccagagacTCTAGATTGG | 82 | 9.6 |
| _COM_2 | | | 105393716 | ATCTTGCTGGCAC | | |
| HsInv397_MLPA | SacI | X | 105393680- | aaaacacaaatccgttgaggttcagaatcccagagacTCTAGATTGG | 82 | 12.4 |
| _COM_2 | | | 105393716 | ATCTTGCTGGCAC | | |
| HsInv403_MLPA | HindIII | X | 75273800- | CTTGAATAAGTGAAATTACTTGCTGGGATGTTTGTCTAGATTGGATC | 83 | 8 |
| _INV | | | 75273833 | TTGCTGGCAC | | |
| HsInv403_MLPA | HindIII | X | 75283891- | AGCTTTGGAAAACTTTGTAATTTTGAGTTGGTCTGACGACTCTAGAT | 84 | 12 |
| _REF | | | 75283930 | TGGATCTTGCTGGCAC | | |
| HsInv790_MLPA | BamHI | 17 | 18433776- | gatccaatccgtagtcttttgtccctcTCTAGATTGGATCTTGCTGG | 85 | 6 |
| _INV | | | 18433802 | CAC | | |
| HsInv790_MLPA | BamHI | 17 | 18433780- | caatccgtagtcttttgtccctcaccTCTAGATTGGATCTTGCTGGC | 86 | 7 |
| _REF | | | 18433805 | AC | | |
| HsInv832_MLPA | BamHI | Y | 16495335- | CTGTGTGATGGAAGAAGGAAACAGAAGAGGTCTAGATTGGATCTTGC | 87 | 2.6 |
| _COM | | | 16495364 | TGGCAC | | |
| HsInv832_MLPA | BamHI | Y | 16495335- | CTGTGTGATGGAAGAAGGAAACAGAAGAGGTCTAGATTGGATCTTGC | 87 | 3 |
| _COM | | | 16495364 | TGGCAC | | |

So far, the iMLPA technique has been developed and tested thoroughly to interrogate 24 human polymorphic inversions flanked by inverted repeats of between 300 bp and 47 kb. This assay has been used already to genotype the inversions in a set of 551 individuals of seven different human populations with an European, African or Asian origin used in the HapMap and 1000 Genome Projects [33]. These populations include individuals with Northern and Western European ancestry (CEU), Toscani (TSI), Yoruba (YRI), Luhya (LWK), Chinese (CHB), Japanese (JPT) and Gujarati Indians (GIH). A total of 12769 genotypes were obtained from the 12957 interrogated. This data corresponds to an estimated genotyping-success rate for the iMLPA technique of 98.5%, ranging between 90.2-100% for the different inversions (Table 3).

TABLE 3
Genotypes obtained by iMLPA for the 24 inversions
in the 551 samples analyzed.

| Inversion ID | REF | HET | INV | ND | TOTAL |
|---|---|---|---|---|---|
| Hsinv389 | 236 | 58 | 253 | 4 | 551 |
| Hsinv124 | 72 | 169 | 306 | 4 | 551 |
| Hsinv340 | 399 | 87 | 43 | 22 | 551 |
| Hsinv209 | 452 | 87 | 8 | 4 | 551 |
| Hsinv278 | 323 | 168 | 54 | 6 | 551 |
| Hsinv344 | 177 | 241 | 117 | 16 | 551 |
| Hsinv393 | 245 | 120 | 182 | 4 | 551 |
| Hsinv379 | 546 | 5 | 0 | 0 | 551 |
| Hsinv790 | 474 | 23 | 0 | 54 | 551 |
| Hsinv031 | 74 | 264 | 210 | 3 | 551 |
| Hsinv045 | 139 | 249 | 155 | 8 | 551 |
| Hsinv055 | 81 | 215 | 237 | 18 | 551 |
| Hsinv397 | 287 | 95 | 166 | 3 | 551 |
| Hsinv374 | 162 | 261 | 125 | 3 | 551 |
| Hsinv114 | 167 | 196 | 185 | 3 | 551 |
| Hsinv030 | 3 | 70 | 478 | 0 | 551 |
| Hsinv061 | 0 | 13 | 534 | 4 | 551 |
| Hsinv832 | 175 | 0 | 106 | 3 | 284 |
| Hsinv396 | 396 | 73 | 74 | 8 | 551 |
| Hsinv341 | 461 | 79 | 4 | 7 | 551 |
| Hsinv347 | 357 | 166 | 25 | 3 | 551 |
| Hsinv403 | 235 | 104 | 207 | 5 | 551 |
| Hsinv040 | 34 | 181 | 333 | 3 | 551 |
| Hsinv072 | 10 | 9 | 529 | 3 | 551 |
| TOTAL | 5505 | 2933 | 4331 | 188 | 12957 |

REF, homozygote for the reference orientation;
HET, heterozygote for the reference and the inverted orientation,
INV, homozygote for the inverted orientation;
ND, not determined.

### EXAMPLE 5

### Comparison of iMLPA Technique and PCR (Regular or Inverse)

On the other hand, in order to calculate the accuracy of the iMLPA assay in front of other methods, we used the genotyping data of 23 of the 24 inversions generated in our laboratory from independent regular or inverse PCR assays (Table 4). In total, we compared 2719 iMLPA genotypes of the 23 inversions in 33-541 individuals with the results obtained by regular PCR or inverse PCR. Only 3 out of the 2719 iMLPA genotypes were not in concordance with those from the PCRs, which allows us to establish the error rate of the iMLPA in approximately 0.1% (Table 5). The errors were distributed among different inversions and apparently were due to a problem with the DNA of the particular individual or the missing of the peak of one orientation in heterozygotes. In all three cases, the iMLPA genotypes were corrected when the iMLPA assay was repeated.

TABLE 4
Genotypes obtained by regular (rPCR) or inverse PCR (iPCR) for 23
inversions in 33-541 samples analyzed.

| Inversion | | | | | | |
|---|---|---|---|---|---|---|
| ID | PCR type | REF | HET | INV | TOTAL | Population |
| HsInv030 | rPCR | 3 | 70 | 468 | 541 | CEU, TSI, |
| | | | | | | YRI, LWK, |
| | | | | | | CHB, JPT, GIH |
| HsInv031 | iPCR | 8 | 44 | 39 | 91 | CEU |
| HsInv040 | iPCR | 5 | 26 | 60 | 91 | CEU |
| HsInv045 | iPCR | 27 | 54 | 10 | 91 | CEU |
| HsInv055 | iPCR | 5 | 30 | 53 | 88 | CEU |
| HsInv061 | iPCR | 0 | 4 | 87 | 91 | CEU |
| HsInv072 | iPCR | 0 | 1 | 90 | 91 | CEU |
| HsInv114 | iPCR | 10 | 31 | 30 | 71 | CEU |
| HsInv124 | iPCR | 28 | 33 | 10 | 71 | CEU |
| HsInv209 | iPCR | 112 | 39 | 4 | 155 | CEU, YRI |
| HsInv278 | iPCR | 57 | 13 | 1 | 71 | CEU |
| HsInv340 | iPCR | 68 | 1 | 0 | 69 | CEU |
| HsInv341 | iPCR | 67 | 3 | 0 | 70 | CEU |
| HsInv344 | iPCR | 13 | 32 | 26 | 71 | CEU |
| HsInv347 | iPCR | 59 | 10 | 2 | 71 | CEU |
| HsInv379 | rPCR | 536 | 5 | 0 | 541 | CEU, TSI, |
| | | | | | | YRI, LWK, |
| | | | | | | CHB, JPT, GIH |
| HsInv389 | iPCR | 52 | 8 | 10 | 70 | CEU |
| HsInv393 | iPCR | 35 | 17 | 16 | 68 | CEU |
| HsInv396 | iPCR | 54 | 8 | 8 | 70 | CEU |
| HsInv397 | iPCR | 53 | 10 | 6 | 69 | CEU |
| HsInv403 | iPCR | 45 | 15 | 11 | 71 | CEU |
| HsInv790 | iPCR | 64 | 0 | 0 | 64 | CEU |
| Hsinv832 | iPCR | 33 | 0 | 0 | 33 | CEU |
| TOTAL | | 1334 | 454 | 931 | 2719 | |

REF, homozygote for the reference orientation;
HET, heterozygote for the reference and the inverted orientation,
INV, homozygote for the inverted orientation.
CEU: individuals with Northern and Western European ancestry;
TSI: individuals with Toscani ancestry;
YRI: individuals with Yoruba ancestry;
LWK: individuals with Luhya ancestry;
CHB: individuals with Chinese ancestry;
JPT: individuals with Japanese ancestry and
GIH: individuals with Gujarati Indians ancestry.

TABLE 5
Summary of comparison between iMLPA and PCR results. Table shows the
breakpoints (BP) used to detect the inverted (INV) and the reference (REF) orientation
by iMLPA and by regular PCR (rPCR) or inverse PCR (iPCR). Among all samples
analyzed only three inversion genotypes were discordant between both methods.

| Inversion | iMLPA | iMLPA | PCR | PCR | | | | |
|---|---|---|---|---|---|---|---|---|
| ID | INV BP | REF BP | INV BP | REF BP | PCR type | Samples | Conc. | Disc. |
| HsInv030 | BD | CD | BD | CD | rPCR | 541 | 541 | 0 |
| HsInv031 | AC | CD | AC | AB | iPCR | 91 | 91 | 0 |
| HsInv040 | BD | AB | AC | AB | iPCR | 91 | 91 | 0 |
| HsInv045 | AC | CD | BD | AB | iPCR | 91 | 91 | 0 |
| HsInv055 | AC | AB | AC | AB | iPCR | 88 | 88 | 0 |
| HsInv061 | BD | AB | BD | CD | iPCR | 91 | 91 | 0 |
| HsInv072 | BD | AB | AC | CD | iPCR | 91 | 91 | 0 |
| HsInv114 | AC | CD | AC | CD | iPCR | 71 | 71 | 0 |
| HsInv124 | BD | CD | BD | CD | iPCR | 71 | 71 | 0 |
| HsInv209 | AC | AB | AC | AB | iPCR | 155 | 155 | 0 |
| HsInv278 | BD | AB | BD | AB | iPCR | 71 | 71 | 0 |
| HsInv340 | BD | AB | BD | AB | iPCR | 69 | 68 | 1 |
| HsInv341 | AC | AB | BD/AC | AB/CD | iPCR | 70 | 70 | 0 |
| HsInv344 | BD | AB | BD | AB | iPCR | 71 | 71 | 0 |
| HsInv347 | AC | AB | AC/BD | AB/CD | iPCR | 71 | 71 | 0 |
| HsInv379 | BD | CD | AC | CD | rPCR | 541 | 541 | 0 |
| HsInv389 | AC | AB | AC | AB | iPCR | 70 | 70 | 0 |
| HsInv393 | BD | AB | AC | AB | iPCR | 68 | 68 | 0 |
| HsInv396 | BD | CD | AC | CD | iPCR | 70 | 69 | 1 |
| HsInv397 | AC | AB | BD | CD | iPCR | 69 | 68 | 1 |
| HsInv403 | AC | CD | AC | CD | iPCR | 71 | 71 | 0 |
| HsInv790 | AC | AB | AC | AB | iPCR | 64 | 64 | 0 |
| Hsinv832 | AC | AB | AC | AB | iPCR | 33 | 33 | 0 |

Conc.: Concordant genotype;
Disc.: Discordant genotype.

In summary, it is described here a new method for improved genotyping of a large number of inversions mediated by inverted repeats through a fast and high-throughput assay. By comparison with other techniques used to genotype inversions one by one, like inverse PCR [13,20], iMLPA has shown a very high sensitivity, reproducibility and accuracy. Besides, iMLPA is the fastest method to determine the inversion genotypes in big sets of samples, being able to produce 12769 genotypes in a short period of time. Finally, this technique could be adapted to the analysis of other structural variants, like translocations, or complex genomic regions in which the exact organization is not clear.

The invention also relates to:

[1]. An in vitro method for detecting in a sample, comprising a plurality of sample nucleic acids of different sequence, the presence of at least one specific genomic inversion structural variant characterized by comprising, at least, the following successive steps:
i. Digesting nucleic acids comprised in the sample with restriction enzymes
ii. Circularization by self-ligation of the digested nucleic acid fragments with ligase enzymes
iii. Breaking nucleic acids obtained in the previous step (ii) and recovery of them by purification
iv. Mixing recovered nucleic acids of previous step (iii) with a plurality of different probe pairs, each probe pair comprising:
   a) A first left nucleic acid oligonucleotide having a first target region complementary to one of the adjacent sequences of the nucleic acid, circularizated by self-ligation, specific of the reference or inverted orientation or common for both orientations
   b) A second right nucleic acid oligonucleotide having a second target region complementary to one of the adjacent sequences of the nucleic acid, circularizated by self-ligation, specific of the reference or inverted orientation or common for both orientations
v. Incubating the plurality of sample nucleic acids with the probe oligonucleotides allowing hybridization of complementary nucleic acids and assembling of the two parts of probe pair that are complementary to the target sequence to form final assembled probes
vi. Amplifying the assembled probes by multiplex PCR, using at least 3 different pairs of universal labeled primers, wherein each pair of primers is formed by a common reverse primer and a specific forward primer in each case labeled with a different labeling compound.
vii. Detecting the amplicon or PCR amplification product

[2]. In vitro method according to [1] wherein nucleic acid is DNA.

[3]. In vitro method according to [1] or [2] wherein at least 24 genomic inversions are detected simultaneously.

[4]. In vitro method according to any of [1] to [3] wherein the inversions detected are flanked by repetitive sequences up to 70 kb.

[5]. In vitro method according to any of [1] to [4] wherein the restriction enzyme is selected among those which generate staggered ends.

[6]. In vitro method according to [5] wherein the restriction enzyme is selected from: EcoRI, HindII, SacI, NsiI, BamHI and BglII, or combinations thereof.

[7]. In vitro method according to any of [1] to [6] wherein the ligase enzyme is T4 DNA Ligase.

[8]. In vitro method according to any of [1] to [7] wherein the probes, additionally to the target region of the sequence hybridizing specifically with their corresponding complementary parts of the DNA samples, also comprise a variable stuffer segment to adjust the probes lengths and a sequence complementary to the forward or reverse universal primers used in multiplex PCR amplification.

[9]. In vitro method according to any of [1] to [8] wherein the probe pairs are selected from SEQ ID No. 1 to SEQ ID NO: 87 or combinations thereof.

[10]. In vitro method according to [9] wherein the left probe is selected from: SEQ ID NO: 1 to SEQ ID NO: 48 or combinations thereof; and the right probe is selected from: SEQ ID NO: 49 to SEQ ID NO: 87, or combinations thereof.

[11]. In vitro method according to any of [1] to [10] wherein the pairs of universal primers are selected from: SEQ ID No. 88 and SEQ ID No. 89; SEQ ID No. 88 and SEQ ID No. 90; SEQ ID No. 88 and SEQ ID No. 91, being SEQ ID No. 88 the common reverse primer and each of SEQ ID No. 89, SEQ ID No. 90 or SEQ ID No. 91, specific forward primers, differentially labeled one from each other.

[12]. In vitro method according to any of [1] to [11] wherein the primers labeling compound is a fluorocrom selected from: FAM, VIC or NED.

[13]. Nucleic acid probe selected from any of SEQ ID No. 1 to SEQ ID No. 87 or mixtures thereof.

[14]. Nucleic acid probe of [13], or mixtures thereof, for use in an in vitro method according to [1] to [12].

[15]. Kit for performing the in vitro method according to [1] to [12], comprising a nucleic acid probe according to [13], or mixtures thereof.

### REFERENCES

1. Thomas, N. S., Bryant, V., Maloney, V., Cockwell, A. E., & Jacobs, P. A., Investigation of the origins of human autosomal inversions. Hum Genet 123 (6), 607-616 (2008).
2. Antonacci, F., Kidd, J. M., Marques-Bonet, T., Ventura, M., Siswara, P., Jiang, Z., & Eichler, E. E., Characterization of six human disease-associated inversion polymorphisms. Hum Mol Genet 18 (14), 2555-2566 (2009).
3. Giglio, S., Calvari, V., Gregato, G., Gimelli, G., Camanini, S., Giorda, R., Ragusa, A., Guerneri, S., Selicorni, A., Stumm, M., Tonnies, H., Ventura, M., Zollino, M., Neri, G., Barber, J., Wieczorek, D., Rocchi, M., & Zuffardi, O., Heterozygous submicroscopic inversions involving olfactory receptor-gene clusters mediate the recurrent t(4;8)(p16;p23) translocation. Am J Hum Genet 71 (2), 276-285 (2002).
4. Szamalek, J. M., Cooper, D. N., Schempp, W., Minich, P., Kohn, M., Hoegel, J., Goidts, V., Hameister, H., & Kehrer-Sawatzki, H., Polymorphic micro-inversions contribute to the genomic variability of humans and chimpanzees. Hum Genet 119 (1-2), 103-112 (2006).
5. Osborne, L. R., Li, M., Pober, B., Chitayat, D., Bodurtha, J., Mandel, A., Costa, T., Grebe, T., Cox, S., Tsui, L. C., & Scherer, S. W., A 1.5 million-base pair inversion polymorphism in families with Williams-Beuren syndrome. Nat Genet 29 (3), 321-325 (2001).
6. Small, K., Iber, J., & Warren, S. T., Emerin deletion reveals a common X-chromosome inversion mediated by inverted repeats. Nat Genet 16 (1), 96-99 (1997).
7. Feuk, L., MacDonald, J. R., Tang, T., Carson, A. R., Li, M., Rao, G., Khaja, R., & Scherer, S. W., Discovery of human inversion polymorphisms by comparative analysis of human and chimpanzee DNA sequence assemblies. PLoS Genet 1 (4), e56 (2005).
8. Korbel, J. O., Urban, A. E., Affourtit, J. P., Godwin, B., Grubert, F., Simons, J. F., Kim, P. M., Palejev, D., Carriero, N. J., Du, L., Taillon, B. E., Chen, Z., Tanzer, A., Saunders, A. C., Chi, J., Yang, F., Carter, N. P., Hurles, M. E., Weissman, S. M., Harkins, T. T. et al., Paired-end mapping reveals extensive structural variation in the human genome. Science 318 (5849), 420-426 (2007).
9. Liu, Q., Nozari, G., & Sommer, S. S., Single-tube polymerase chain reaction for rapid diagnosis of the inversion hotspot of mutation in hemophilia A. Blood 92 (4), 1458-1459 (1998).
10. Pang, A. W., Migita, O., Macdonald, J. R., Feuk, L., & Scherer, S. W., Mechanisms of formation of structural variation in a fully sequenced human genome. Hum Mutat 34 (2), 345-354 (2013).
11. Rossetti, L. C., Radic, C. P., Abelleyro, M. M., Larripa, I. B., & De Brasi, C. D., Eighteen Years of Molecular Genotyping the Hemophilia Inversion Hotspot: From Southern Blot to Inverse Shifting-PCR. Int J Mol Sci 12 (10), 7271-7285 (2011).
12. Turner, D. J., Shendure, J., Porreca, G., Church, G., Green, P., Tyler-Smith, C., & Hurles, M. E., Assaying chromosomal inversions by single-molecule haplotyping. Nat Methods 3 (6), 439-445 (2006).
13. Rossetti, L. C., Radic, C. P., Larripa, I. B., & De Brasi, C. D., Genotyping the hemophilia inversion hotspot by use of inverse PCR. Clin Chem 51 (7), 1154-1158 (2005).
14. Turner, D. J., Tyler-Smith, C., & Hurles, M. E., Long-range, high-throughput haplotype determination via haplotype-fusion PCR and ligation haplotyping. Nucleic Acids Res 36 (13), e82 (2008).
15. Ochman, H., Gerber, A. S., & Hartl, D. L., Genetic applications of an inverse polymerase chain reaction. Genetics 120 (3), 621-623 (1988).
16. Pavlopoulos, A., Identification of DNA sequences that flank a known region by inverse PCR. Methods Mol Biol 772, 267-275 (2011).
17. Saitsu, H., Osaka, H., Sugiyama, S., Kurosawa, K., Mizuguchi, T., Nishiyama, K., Nishimura, A., Tsurusaki, Y., Doi, H., Miyake, N., Harada, N., Kato, M., & Matsumoto, N., Early infantile epileptic encephalopathy associated with the disrupted gene encoding Slit-Robo Rho GTPase activating protein 2 (SRGAP2). Am J Med Genet A 158A (1), 199-205 (2012).
18. Thorsen, J., Micci, F., & Heim, S., Identification of chromosomal breakpoints of cancer-specific translocations by rolling circle amplification and long-distance inverse PCR. Cancer Genet 204 (8), 458-461 (2011).
19. Peng, Z., Zhao, Z., Nath, N., Froula, J. L., Clum, A., Zhang, T., Cheng, J. F., Copeland, A. C., Pennacchio, L. A., & Chen, F., Generation of long insert pairs using a Cre-LoxP Inverse PCR approach. PLoS One 7 (1), e29437 (2012).
20. Rossetti, L. C., Radic, C. P., Larripa, I. B., & De Brasi, C. D., Developing a new generation of tests for genotyping hemophilia-causative rearrangements involving int22h and Int1h hotspots in the factor VIII gene. J Thromb Haemost 6 (5), 830-836 (2008).
21. Abou-Elew, H., Ahmed, H., Raslan, H., Abdelwahab, M., Hammoud, R., Mokhtar, D., & Arnaout, H., Genotyping of intron 22-related rearrangements of F8 by inverse-shifting PCR in Egyptian hemophilia A patients. Ann Hematol 90 (5), 579-584 (2011).
22. Fujita, J., Miyawaki, Y., Suzuki, A., Maki, A., Okuyama, E., Murata, M., Takagi, A., Murate, T., Suzuki, N., Matsushita, T., Saito, H., & Kojima, T., A possible mechanism for Inv22-related F8 large deletions in severe hemophilia A patients with high responding factor VIII inhibitors. J Thromb Haemost 10 (10), 2099-2107 (2012).
23. He, Z. H., Chen, S. F., Chen, J., & Jiang, W. Y., A modified I-PCR to detect the factor VIII Inv22 for genetic diagnosis and prenatal diagnosis in haemophilia A. Haemophilia 18 (3), 452-456 (2012).
24. WO2001/61033 A2 (SCHOUTEN, J. P.) 15 Feb. 2001.
25. Redeker, E. J., de Visser, A. S., Bergen, A. A., & Mannens, M. M., Multiplex ligation-dependent probe amplification (MLPA) enhances the molecular diagnosis of aniridia and related disorders. Mol Vis 14, 836-840 (2008).
26. Taylor, C. F., Charlton, R. S., Burn, J., Sheridan, E., & Taylor, G. R., Genomic deletions in MSH2 or MLH1 are a frequent cause of hereditary non-polyposis colorectal cancer: identification of novel and recurrent deletions by MLPA. Hum Mutat 22 (6), 428-433 (2003).
27. Armengol, L., Villatoro, S., Gonzalez, J. R., Pantano, L., Garcia-Aragones, M., Rabionet, R., Caceres, M., & Estivill, X., Identification of copy number variants defining genomic differences among major human groups. PLoS One 4 (9), e7230 (2009).
28. Volikos, E., Robinson, J., Aittomaki, K., Mecklin, J. P., Jarvinen, H., Westerman, A. M., de Rooji, F. W., Vogel, T., Moeslein, G., Launonen, V., Tomlinson, I. P., Silver, A. R., & Aaltonen, L. A., LKB1 exonic and whole gene deletions are a common cause of Peutz-Jeghers syndrome. J Med Genet 43 (5), e18 (2006).
29. Procter, M., Chou, L. S., Tang, W., Jama, M., & Mao, R., Molecular diagnosis of Prader-Willi and Angelman syndromes by methylation-specific melting analysis and methylation-specific multiplex ligation-dependent probe amplification. Clin Chem 52 (7), 1276-1283 (2006).
30. Wehner, M., Mangold, E., Sengteller, M., Friedrichs, N., Aretz, S., Friedl, W., Propping, P., & Pagenstecher, C., Hereditary nonpolyposis colorectal cancer: pitfalls in deletion screening in MSH2 and MLH1 genes. Eur J Hum Genet 13 (8), 983-986 (2005).
31. Hochstenbach, R., Meijer, J., van de Brug, J., Vossebeld-Hoff, I., Jansen, R., van der Luijt, R. B., Sinke, R. J., Page-Christiaens, G. C., Ploos van Amstel, J. K., & de Pater, J. M., Rapid detection of chromosomal aneuploidies in uncultured amniocytes by multiplex ligation-dependent probe amplification (MLPA). Prenat Diagn 25 (11), 1032-1039 (2005).
32. Pantano, L., Armengol, L., Villatoro, S., & Estivill, X., ProSeeK: a web server for MLPA probe design. BMC Genomics 9, 573 (2008).
33. Altshuler, D. M., Gibbs, R. A., Peltonen, L., Dermitzakis, E., Schaffner, S. F., Yu, F., Bonnen, P. E., de Bakker, P. I., Deloukas, P., Gabriel, S. B., Gwilliam, R., Hunt, S., Inouye, M., Jia, X., Palotie, A., Parkin, M., Whittaker, P., Chang, K., Hawes, A., Lewis, L. R. et al., Integrating common and rare genetic variation in diverse human populations. Nature 467 (7311), 52-58 (2010).

1. An in vitro method for detecting the orientation of a genomic sequence within a larger sequence, wherein said genomic sequence is connected to the larger sequence at its 5′ and 3′ ends by a 5′ junction region and by a 3′ junction region in a sample comprising nucleic acids, said method comprising the following steps:
(i) digesting nucleic acids with at least a restriction enzyme, said restriction enzyme having at least a target site in the genomic sequence flanked by a junction region and at least another target site outside the genomic sequence flanked by a junction region,
(ii) circularizing the digested nucleic acid fragments obtained in step (i) by self-ligation with a ligase enzyme, thereby generating a circular nucleic acid comprising a junction region and a reconstituted target site for the restriction enzyme used in step (i), said reconstituted target site flanked on one side by the region originally located 3′ with respect to the junction region and on the other side by the region originally located 5′ with respect to the junction region,
(iii) incubating the circularized nucleic acids obtained in step (ii) with at least a probe pair, each probe pair selected from the group consisting of:
I. a probe pair comprising:
a) a first oligonucleotide Having a 5′ region and a 3′ region, wherein the 3′ region of said first oligonucleotide is complementary to a region of the genomic sequence flanked by a junction region and wherein the 3′ end of said first oligonucleotide is phosphorylated and
b) a second oligonucleotide having a 5′ region and a 3′ region, wherein the 5′ region of said second oligonucleotide is complementary to a region of the larger sequence originally located outside the genomic sequence flanked by a junction region
and wherein the nucleotide position within the circularized genomic sequence to which the 3′ end of the first oligonucleotide hybridizes and the nucleotide position within the genomic sequence to which the 5′ end of the second oligonucleotide hybridizes are adjacent positions, and
wherein the region of the circularized genomic sequence to which the first and second oligonucleotide hybridize comprises the target site generated after the ligation step (ii), and
II. a probe pair comprising:
a) a first oligonucleotide having a 5′ region and a 3′ region, wherein the 3′ region of said first oligonucleotide is complementary to a region of the genomic sequence originally located outside the genomic sequence flanked by a junction region and wherein the 3′ end of said first oligonucleotide is phosphorylated and
b) a second oligonucleotide having a 5′ region and a 3′ region, wherein the 5′ region of said second oligonucleotide is complementary to a region of the genomic sequence flanked by a junction region
and wherein the nucleotide position within the circularized genomic sequence to which the 3′ end of the first oligonucleotide hybridizes and the nucleotide position within the genomic sequence to which the 5′ end of the second oligonucleotide hybridizes are adjacent positions, and
wherein the region of the circularized genomic sequence to which the first and second oligonuclectide hybridize comprises the target site generated after the ligation step (ii),
(iv) ligating the 3′ end of the first oligonucleotide with the 5′ end of the second oligonuclectide of each probe pair to form an assembled probe,
(v) amplifying the assembled probe obtained in step (iv) by using a pair of primers, wherein the forward primer hybridizes to the 5′ region of the first oligonucleotide of the probe pair and the reverse primer hybridizes to the 3′ region of the second oligonucleotide of the probe pair, and
(vi) detecting the product of step (v).

2. The in vitro method according to claim 1, wherein the restriction enzyme target site outside the _{g}enomic sequence flanked by a junction region is located in a junction region or is located outside the junction region.

3. (canceled)

4. The in vitro method according to claim 1, wherein the 5′ junction region and/or the 3′ junction region is an inverted repeat sequence.

5. The in vitro method according to claim 4, wherein if the 5′ junction region and the 3′ junction region are inverted repeat sequences, both are the same inverted repeat sequence.

6. The in vitro method according to claim 1, wherein after step (ii) the nucleic acids are broken and recovered by purification.

7. The in vitro method according to claim 1, wherein a plurality of different probe pairs is used and wherein the 5′ region of the first oligonucleotide of each probe pair contains a nucleotide sequence of different length between the sequence complementary to the forward primer used in step (v) and the 3′ region of the first oligonucleotide.

8. The in vitro method according to claim 1, wherein a plurality of different probe pairs is used and wherein the 3′ region of the second oligonucleotide of each probe pair contains a nucleotide sequence of different length between the sequence complementary to the reverse primer used in step (v) and the 5′ region of the second oligonucleotide.

9. The in vitro method according to claim 1, wherein the adjacent positions to which the 3′ and of the first oligonucleotide and the 5′ end of the second oligonucleotide hybridize are comprised within the target site generated after the ligation step (ii).

10. The in vitro method according to claim 1, wherein the ligase enzyme used in step (ii) is T4 DNA ligase and/or wherein the ligase enzyme used in step (iv) is a NAD-dependent ligase enzyme.

11. (canceled)

12. The in vitro method according to claim 1, wherein the forward primer is labeled.

13. The in vitro method according to claim 12, wherein a plurality of pairs of primers is used in step (v) and wherein the forward primer of each pair is labeled with a different compound, and wherein optionally the labeling compound is selected from the group consisting of FAM, VIC, HEX/PET, TAMPA and NED.

14. The in vitro method according to claim 1, wherein the reverse primer is labeled.

15. The in vitro method according to claim 14, wherein a plurality of pairs of primers is used in step (v) and wherein the reverse primer of each pair is labeled with a different compound, and wherein optionally the labeling compound is selected from the group consisting of FAM, VIC, HEX/PET, TAMRA and NED.

16. (canceled)

17. The in vitro method according to claim 1, wherein the nucleic acid is DNA.

18. The in vitro method according to claim 4, wherein each inverted repeat sequence has up to 70 kb.

19. The in vitro method according to claim 1, wherein the restriction enzyme is a restriction enzyme generating staggered ends.

20. (canceled)

21. The in vitro method according to claim 1 wherein the probe pair is selected from the group consisting of SEQ ID NO: 1 to SEQ ID NO: 87 or combinations thereof.

22. The in vitro method according to claim 21, wherein
(i) the first oligonucleotide of the probe pair is selected from the group consisting of SEQ ID NO: 1 to SEQ ID NO: 48 or combinations thereof; and the second oligonucleotide of the prone pair is selected from the group consisting of SEQ ID NO: 49 to SEQ ID NO: 87 or combinations thereof and/or
(ii) wherein the pair of primers used in step (v) is selected from the group consisting of SEQ ID NO: 98 and SEQ ID NO: 89; SEQ ID NO: 88 and SEQ ID NO: 90; SEQ ID NO: 88 and SEQ ID NO: 91, being SEQ ID NO: 88 the reverse primer and each of SEQ ID NO: 89, SEQ ID NO: 90 or SEQ ID NO: 91 the forward primer.

23. (canceled)

24. An oligonucleotide probe selected from the group consisting of any of SEQ ID NO: 1 to SEQ ID NO: 87 or mixtures thereof.

25. Kit comprising an oligonucleotide probe pair, wherein the first oligonuclectide of the probe pair is selected from the group consisting of SEQ ID NO: 1 to SEQ ID NO: 48 or combinations thereof; and the second oligonucleotide of the probe pair is selected from the group consisting of SEQ ID NO: 49 to SEQ ID NO: 87 or combinations thereof.

26-31. (canceled)
